(19) United States
(12) Patent Application Publication
(10) Pub. No.: US 2004/0052845 A1
(43) Pub. Date: Mar. 18, 2004

(54) Hydrogel-driven drug dosage form

(76) Inventors: Leah E Appel, Bend, OR (US); Walter C Babcock, Bend, OR (US); Ronald A Beyerinck, Bend, OR (US); Mark B Chidlaw, Bend, OR (US); William J Curatolo, Niantic, CT (US); Dwayne T Friesen, Bend, OR (US); Scott M Herbig, East Lyme, CT (US); Avinash G Thombre, East Lyme, CT (US)

Correspondence Address: PFIZER INC., PATENT DEPARTMENT, MS8260-1611, EASTERN POINT ROAD, GROTON, CT, 06340, US

(21) Appl. No.: 10/344,171

(22) PCT Filed: Aug. 3, 2001
(86) PCT No.: PCT/IB01/01390

(51) Int. Cl. A61K 9/24

(52) U.S. Cl. 424/471

(57) ABSTRACT

A controlled release dosage form has a coated core with the core comprising a drug-containing composition and a water-swellable composition, each occupying separate regions within the core. The coating around the core is water-permeable, water-insoluble and has at least one delivery port therethrough. A variety of geometric arrangements are disclosed.

### BACKGROUND OF THE INVENTION

The present invention relates to a dosage form that provides a controlled release of a beneficial agent, or drug, to an environment of use.

Osmotic and hydrogel-driven drug delivery devices for the release of a drug have been known in the art for some time. Exemplary dosage forms have included a tablet comprising a semipermeable wall surrounding a compartment containing the drug and a layer of swellable hydrogel, with the drug being delivered through a passageway in the semipermeable wall by swelling of the hydrogel, as described in U.S. Pat. No. 4,327,725; another tablet comprising a wall permeable to an exterior fluid but impermeable to the drug, the wall surrounding a compartment containing two osmotic agents, two expandable polymers and the drug, as described in U.S. Pat. No. 4,612,008; drug dispersed in a swellable hydrogel matrix core that releases the drug by diffusion into the environment of use, as described in U.S. Pat. No. 4,624,848; a hydrogel reservoir containing a multiplicity of tiny pills wherein each tiny pill consists of a wall surrounding a drug core, as described in U.S. Pat. No. 4,851,232; and a two-layered tablet wherein one layer is drug mixed with a hydrogel and the other layer is a hydrogel, as described in U.S. Pat. No. 5,516,527.

While the conventional dosage forms described above are functional, nonetheless such dosage forms suffer from a variety of drawbacks. A controlled release dosage form should ideally deliver substantially all of the drug from the dosage form to the environment of use. However, a common problem encountered by osmotic and hydrogel-driven dosage forms, particularly when the drug has low aqueous solubility, is that residual drug is left in the tablet interior after the hydrogel or other swellable material has completely swelled. This residual drug is not available for absorption and, accordingly, such dosage forms require increased amounts of drug to compensate for the failure of the system to release all of the drug into the environment of use.

In addition, the controlled release dosage form must operate within certain size constraints, and yet be capable of delivering most or all of the drug to the environment of use. Dosage forms, particulary for humans, are limited in size, and are usually less than 1 gram, more preferably less than 700 mg in weight. However, for some types of drugs, the dose amount may make up to half or even more of the weight of the dosage form. The water-swellable materials that provide th delivery of the drug must in instances where the dose is high be capable of providing a highly efficient delivery of the drug, since very little of the dosage form may be available for the swellable material or other excipients.

In addition, it is often desired that the dosage form begin extruding drug relatively quickly upon entering the use environment. However, many delivery systems exhibit a time lag before extruding drug. This can be particularly problematic when the drug has low aqueous solubility or is hydrophobic. Several techniques have been proposed to reduce the time lag, but each has its own drawback. One technique has been to provide high-permeability coatings by utilizing thin coatings around the dosage form. While this technique provides a quicker uptake of fluid, the thin coating lacks strength and often bursts in use or provides insufficient protection to the dosage form which becomes susceptible to damage during handling. Yet another technique has involved providing pores or one or more passageways that communicate with the water-swellable materials, but this often leads to unacceptable amounts of residual drug. Another technique involves coating the dosage form with an immediate release drug formulation, but this requires additional processing steps and provides a dosage form with two different release rates, which may be undesirable.

Yet another problem encountered with conventional osmotic and hydrogel-driven drug delivery systems is that such dosage forms often require the presence of osmagents. Osmagents are selected such that they generate an osmotic pressure gradient across the barrier of the surrounding coating. The osmotic pressure gradient drives the permeation of water into the tablet and the resulting buildup of sufficient hydrostatic pressure, which forces the drug through the delivery port. These osmagents increase the weight of the dosage form, thus limiting the amount of drug which may be contained in the dosage form. In addition, the presence of additional ingredients in the dosage form, such as osmagents, increases the costs of manufacture due to the need to insure uniform concentrations of the ingredients throughout the dosage form, and may have other drawbacks such as adverse effects on compression properties and on drug stability.

Very little has been done to investigate the delivery of drugs from dosage forms having different arrangements of materials. Dosage forms of the prior art generally fall into one of three arrangements. The first is the conventional bilayer design, which is characterized by a drug-containing layer and a water-swellable layer. Exemplary of these devices is Wong, et al., U.S. Pat. No. 4,612,008.

Yet another arrangement consists of a water-swellable layer surrounded by a drug-containing composition. Such a device is shown in Curatolo, U.S. Pat. No. 5,792,471.

Yet another arrangement is shown by McClelland et al., U.S. Pat. No. 5,120,548, which discloses a controlled release delivery device containing swelling modulators blended within swellable polymers.

Nevertheless, there is still a need in the art for a controlled release dosage form that results in a highly efficient delivery of drug to an environment of use with very little residual drug, that allows large drug loading so as to minimize the dosage size, that begins releasing drug soon after entering the environment of use, and that limits the number of necessary ingredients. These needs and others which will become apparent to one skilled in the art are met by the present invention, which is summarized and described in detail below.

### BRIEF SUMMARY OF THE INVENTION

The various aspects of the invention each provide a controlled release drug dosage form for delivery of at least one drug. A first aspect of the invention provides a controlled release drug dosage form comprising a core and a coating around the core. The core comprises a first drug-containing composition, a second drug containing composition, and a water-swellable composition, each occupying separate regions within the core. The water-swellable composition is located between the first and second drug-containing compositions. The coating is water-permeable, water-insoluble, and has at least one delivery port for communication with the first drug-containing composition and at least one additional delivery port for communication with the second drug-containing composition.

A second aspect of the invention provides a controlled release drug dosage form comprising a core and a coating around said core. The core comprises a drug-containing composition and a water-swellable composition, each occupying separate regions within said core. The drug-containing composition surrounds the water-swellable composition. The drug-containing composition comprises a low-solubility drug and a drug-entraining agent. The water-swellable composition comprises a swelling agent. The coating is water-permeable, water-insoluble, and has at least one delivery port therethrough.

A third aspect of the invention provides a controlled release drug dosage form comprising a core and a coating. The core comprises a drug-containing composition and a water-swellable composition, each occupying separate regions within the core. The water-swellable composition comprises a plurality of granules. The drug-containing composition comprises a drug and a drug-entraining agent. The water-swellable composition comprises a swelling agent. The coating is water-permeable, water-insoluble, and has at least one delivery port therethrough.

A fourth aspect of the invention provides a controlled release drug dosage form comprising a core and a coating. The core is substantially homogeneous throughout and comprises a mixture of a drug, a drug-entraining agent, a fluidizing agent, and a swelling agent. The coating is water-permeable, water-insoluble, and has at least one delivery port therethrough.

This invention further provides a method of treating a disease or condition amenable to treatment with a pharmaceutical agent which is administered in a controlled release (i.e., sustained release or delayed release) dosage form, comprising administering to a person in need of such treatment a controlled release dosage form according to any of the four aspects disclosed above, said dosage form comprising an effective amount of said pharmaceutical agent.

The amount of a particular compound which is administered will necessarily be varied according to principles well known in the art taking into account factors such as the particular compound of interest, the severity of the disease or condition being remediated and the size and age of the patient. In general, the compound will be administered so that an effective dose is received, an “effective dose” being determined from safe and efficacious ranges of administration already known for the particular compound of interest. Alternatively, an effective amount can be determined by the attending physician.

The methods of treatment disclosed above are not limited by or to any particular disease or indication, and the scope of such methods is intended to be broad, such methods of treatment including, but not being limited to, any of the classes of compounds or specific compounds disclosed hereinbelow.

The various aspects of the present invention have one or more of the following advantages. The dosage forms of the present invention are capable of delivering greater amounts of drug to the desired environment of use with greater efficiency using smaller amounts of swelling materials, and also result in lower amounts of residual drug than do conventional compositions. The compositions are also capable of higher drug loading compared with conventional compositions. In addition, the compositions begin delivering drug to the environment of use more quickly than do conventional dosage forms. The dosage forms are capable of rapidly delivering a drug without the coating failing due to rupture as a result of excessive pressure within the core when the dosage form is introduced into an environment of use.

In addition, the various embodiments provide at least one manufacturing advantage relative to the bi-layer design, in that the location of the delivery port is not as important, as discussed below. In addition, for the aspect comprising a homogeneous core, that embodiment eliminates processing associated with forming separate layers.

The foregoing and other objectives, features, and advantages of the invention will be more readily understood upon consideration of the following detailed description of the invention, taken in conjunction with the accompanying drawings.

### BRIEF DESCRIPTION OF THE DRAWING

FIGS. 1-4 are schematic drawings of cross sections of exemplary embodiments of dosage forms of the present invention.

### DETAILED DESCRIPTION OF THE INVENTION

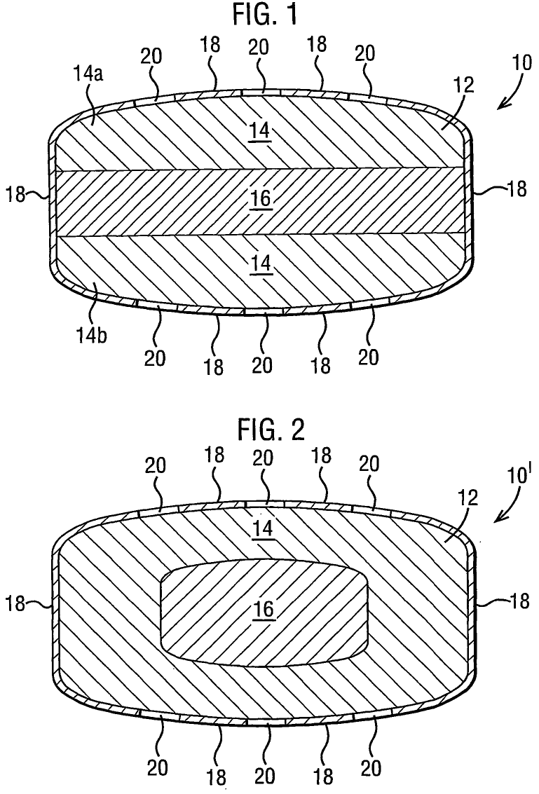

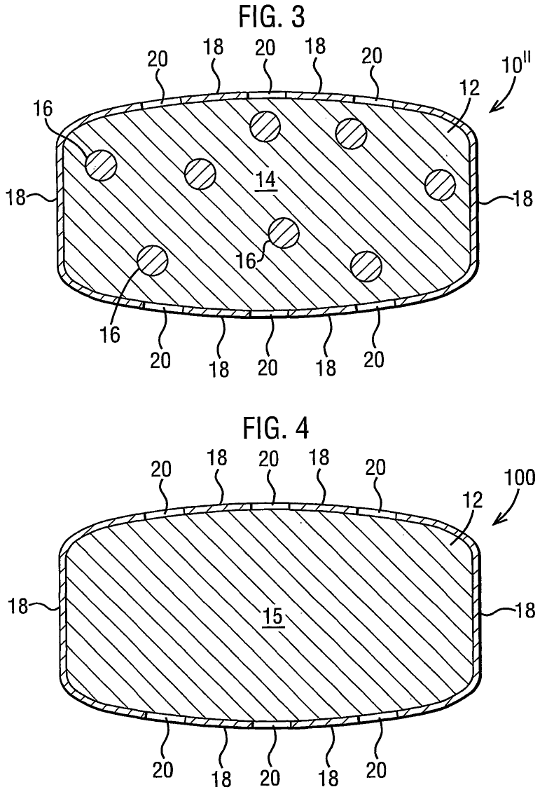

The present invention provides a controlled release dosage form that is specifically designed to provide controlled release of at least one drug primarily by imbibition of water and extrusion of drug from the dosage form as opposed to primarily by diffusion. Referring now to the figures, wherein like numerals refer to like elements, FIGS. 1-4 depict schematically four exemplary dosage form arrangements. FIG. 1 depicts a “tri-layer” tablet; FIG. 2 depicts a “concentric core” tablet; FIG. 3 depicts a “granular core” tablet; and FIG. 4 depicts a “homogeneous core” tablet. Certain features common to all of the exemplary embodiments may be understood by first considering FIG. 1 which shows an exemplary tri-layer dosage form 10 having a core 12 comprising drug-containing composition(s) 14 and a water-swellable composition 16. The drug-containing composition(s) and the water-swellable composition occupy separate regions in the core. By “separate regions” is meant that the two compositions occupy separate volumes, such that the two are not substantially mixed together. Of course, a small amount of intermixing of the compositions may occur where the compositions come in contact with each other, for example, at the interface between two layers. A coating 18 surrounds the core 12 and is water-permeable, water-insoluble and has one or more delivery ports 20 therethrough. In use, the core 12 imbibes water through the coating 18 from the environment of use such as the gastrointestinal (“GI”) tract of a mammal. The imbibed water causes the water-swellable composition 16 to swell, thereby increasing the pressure within the core 12. The imbibed water also increases the fluidity of the drug-containing composition. The pressure difference between the core 12 and the environment of use drives the release of the fluidized drug-containing composition(s) 14. Because the coating 18 remains intact, the drug-containing composition(s) 14 are extruded out of the core 12 through the delivery port(s) 20 into the environment of use. Because the water-swellable composition 16 contains no drug, almost all of the drug is extruded through the delivery port(s) 20, l aving very little residual drug.

The dosage form of the present invention releases the drug to an environment of use primarily by “extrusion” rather than by diffusion. The term “extrusion” as used herein is intended to convey an expulsion or forcing out of some or all of the drug through one or more delivery ports or pores in the coating to the exterior of the dosage form by hydrostatic forces, to be distinguished from delivery by a diffusion mechanism or by erosion of the mass of the device. The drug may be released primarily by extrusion either in the form of a suspension of solids in aqueous solution or the drug may be in solution, to the extent dissolution has taken place in the core 12.

Reference to the “release” of drug as used herein means (1) transport of drug from the interior of the dosage form to its exterior such that it contacts fluid within a mammal (e.g., a mammal's GI tract) following delivery or (2) transport of drug from the interior of the dosage form such that it contacts a test medium for evaluation of the dosage form by an in vitro test as described below. Reference to a Ruse environments can thus be either to in vivo fluids or to an in vitro test medium. “Introduction” to a use environment includes either by ingestion or swallowing or use of implants or suppositories, where the use environment is in vivo, or being placed in a test medium where the use environment is in vitro.

### Dosage Form Arrangement

Four exemplary dosage form arrangements are schematically shown in FIGS. 1-4.

FIG. 1 depicts a “tri-layer” tablet 10 comprising a core 12 that has two drug-containing compositions 14a and 14b on either side of a water-swellable composition 16 and, surrounding the core 12, a coating 18 that has at least one delivery port 20 through the coating connecting each drug layer 14a and 14b with the exterior of the dosage form. The tri-layer dosage form provides several advantages. First, the dosage form may be used to deliver two different drugs. Thus, the drug-containing composition 14a may contain a drug that is different than the drug in drug-containing composition 14b. Second, even when the drug-containing compositions 14a and 14b contain the same drug, the two drug-containing compositions may be formulated differently so as to provide different release rates for the drug. Thus, for example, drug-containing composition 14a could provide a fast release rate for a drug, while drug-containing composition 14b could provide a slow release rate, thus allowing a wide range of drug profiles to be achieved.

Another advantage of the tri-layer design is that the delivery port is located on both sides of the core, rather than on a single side as in the bi-layer arrangement. It is desir d that the bi-layer dosage form have at least one delivery port in communication with the drug-containing composition; A problem when manufacturing bi-layer dosage forms is that for some compositions, providing a delivery port in communication with the water-swellable composition diminishes performance. Thus, care and added expense are required during manufacturing to locate the side of the dosage form facing the drug-containing composition and then provide a delivery port only on that side of the dosage form. In contrast, for the tri-layer design, it is desired to have a delivery port on both sides of the dosage-form. Therefore, it is no longer necessary to locate the correct side for providing the delivery port, since a delivery port is provided on both sides of the dosage form.

FIG. 2 depicts a “concentric core” tablet 10′ comprising a core 12 that has a drug-containing composition 14 that surrounds a water-swellable composition 16 and surrounding the core, a coating 18 that has at least one delivery port 20 through the coating 18 connecting the drug layer 14 with the exterior of the dosage form. The concentric core dosage form provides at least one processing advantage relative to the bi-layer arrangement in that the location of the delivery port is not critical, since the water-swellable composition is surrounded by the drug-containing composition. Thus any delivery port will be in communication with the drug-containing composition regardless of location. Also, water must pass through the drug-containing composition prior to entering the water-swellable composition ensuring that the drug-containing composition is fluid enough to be delivered prior to pressure being exerted by the water-swellable composition.

FIG. 3 depicts a “granular core” tablet 10″ comprising a core 12, a coating 18 and at least one delivery port 20. The core comprises a drug-containing composition 14, and multiple granules of a water-swellable composition 16 mixed throughout the drug-containing composition 14. Like the concentric core embodiment, the location of the delivery port for the granular core is not important, and therefore provides a manufacturing advantage relative to the bi-layer arrangement.

Yet another advantage of the granular core tablet is that it may be formed using conventional single-layer tablet-manufacturing equipment. This avoids the expense of a multi-layer tablet press.

FIG. 4 depicts a “homogeneous core” tablet 100, comprising a core 12, a coating 18 and at least one delivery port 20. The core comprises a homogenous drug-containing composition 15 that contains both the drug and the swelling materials. The homogeneous core provides at least three manufacturing advantages. First, the location of the delivery port is not important, since any delivery port will be in communication with the drug-containing composition. Second, only a single drug-containing composition needs to be prepared, rather than separate drug-containing compositions and water-swellable compositions. Third, standard singl-layer tablet-making equipment can be used to form the core. Accordingly, the cost associated with preparing additional compositions is eliminated.

### Release Characteristics

An important attribute of the dosage forms of the present invention is the delivery of drug to a use environment in a controlled manner. For some aspects of the present invention, the dosage forms start releasing drug soon after introduction to the use environment. When a rapid onset of delivery is desired, preferably the dosage forms release at least 5 wt % of the drug, and more preferably at least 10 wt % of the drug within 2 hours after introduction to the use environment, where these percentages correspond to the mass of drug released from the core relative to the total mass of drug originally present in the core. By quickly beginning the release of the drug, the dosage form shortens the time required to achieve an effective drug concentration in a use environment such as the upper GI tract. Rapid release can also reduce the time required to achieve an effective drug level in the blood.

It is also desired that the dosage forms release the drug in a controlled manner, preferably at a substantially constant rate. For many drugs, it is preferred that the dosage forms release no more than about 60 wt % of the drug, and more preferably no more than about 50 wt % of the drug, into the use environment within 2 hours after introduction to the use-environment. The rate of release of drug from the dosage form should also be sufficiently high to allow release of the drug within a time frame that allows a substantial fraction of the drug delivered to be absorbed into the blood stream. For many drugs the dosage forms preferably release at least 60 wt % of the drug, and more preferably at least 70 wt % of the drug to the use environment within 16 hours after introduction to the use environment. The inclusion of a fluidizing agent in the drug-containing composition is particularly useful when more rapid delivery of drug to the use environment is desired. In particular, when it is desirable to deliver at least 70 wt % of the drug to the use environment within 12 hours after introduction thereto, the invention allows rapid drug release without rupture or otherwise failure of the dosage form coating during operation.

It is also desired that the dosage forms release a substantial amount of the drug contained within the dosage form, leaving a relatively small residual amount of drug after 24 hours. Obtaining low residual amounts of drug is particularly difficult wh n it is desired to deliver high doses of low-solubility drug. Preferably, the dosage forms of the present invention release at least 80 wt/o of drug, more preferably at least 90 wt %, and even more preferably at least 95 wt % of drug to the use environment within 24 hours after introduction of the dosage form to the use environment.

An in vitro test may be used to determine the release profile(s) of the dosage forms of the present invention. In vitro tests are well known in the art. An example is a “residual test,” which is described below for sertraline HCl. One or more dosage forms is first placed into a stirred USP type 2 dissoette flask containing 900 mL of a buffer solution simulating gastric environment (10 mM HCl, 120 mM NaCl, pH 2.0, 261 mOsm/kg) at 37 □C for 2 hours, then removed, rinsed with deionized water, and transferred to a stirred USP type 2 dissoette flask containing 900 mL of a buffer solution simulating the contents of the small intestine (6 mM KH₂PO₄, 64 mM KCl, 35 mM NaCl, pH 7.2, 210 mOsm/kg). In both flasks, the dosage forms are placed in a wire support to keep the dosage forms off of the bottom of the flask, so that all surfaces are exposed to the moving release solution and the solutions are stirred using paddles that rotate at a rate of 50 rpm. At each time interval, a single dosage form is removed from the solution, released material is removed from the surface, and the dosage form cut in half and placed in 100 mL of a recovery solution (1:1 wt/wt ethanol:water, pH adjusted to 3 with 0.1 N HCl), and vigorously stirred overnight at ambient temperature to dissolve the drug remaining in the dosage form. Samples of the recovery solution containing the dissolved drug are filtered using a Gelman Nylon® Acrodisc®13, 0.45 μm pore size filter, and placed in a vial and capped. Residual drug is analyzed by HPLC. Drug concentration is calculated by comparing UV absorbance of samples to the absorbance of drug standards. The amount remaining in the tablets is subtracted from the total drug present prior to release to obtain the amount released at each time interval.

An alternative in vitro test is a direct test, in which samples of the dosage form are placed into a stirred USP type 2 dissoette flask containing 900 mL of a receptor solution such as USP sodium acetate buffer (27 mM acetic acid and 36 mM sodium acetate, pH 4.5) or 88 mM NaCl. Samples are taken at periodic intervals using a VanKel VK8000 autosampling dissoette with automatic receptor solution replacement. Tablets are placed in a wire support as above, paddle height is adjusted, and the dissoette flasks stirred at 50 rpm at 37 □C. The autosampler dissoette device is programmed to periodically remove a sample of the receptor solution, and the drug concentration is analyzed by HPLC using the procedure outlined above. Since the drug is usually extruded from the dosage form as a suspension in an entraining polymer, there is often a time lag between when the drug is released and when it is dissolved in the test medium, and thus, measured in the direct test. This time lag depends on the solubility of the drug, the test medium, and the ingredients of the drug-containing composition, but typically is on the order of 30 to 90 minutes.

While particular buffers or test media in which to conduct in vitro tests have been described above, any conventional test media may be used as-is well known in the art.

Alternatively, an in vivo test may be used. However, due to the inherent difficulties and complexity of the in vivo procedure, it is preferred that in vitro procedures be used to evaluate dosage forms even though the ultimate use environment is often the human GI tract. Drug dosage forms are dosed orally to a group of mammals, such as humans or dogs and drug release and drug absorption is monitored either by (1) periodically withdrawing blood and measuring the serum or plasma concentration of drug or (2) measuring the amount of drug remaining in the dosage form following its exit from the anus (residual drug) or (3) both (1) and (2). In the second method, residual drug is measured by recovering the tablet upon exit from the anus of the test subject and measuring the amount of drug remaining in the dosage form using the same procedure described above for the in vitro residual test. The difference between the amount of drug in the original dosage form and the amount of residual drug is a measure of the amount of drug released during the mouth-to-anus transit time. This test has limited utility since it provides only a single drug release time point but is useful in demonstrating the correlation between in vitro and in vivo release.

In one in vivo method of monitoring drug release and absorption, the serum or plasma drug concentration is plotted along the ordinate (y-axis) against the blood sample time along the abscissa α-axis). The data may then be analyzed to determine drug release rates using any conventional analysis, such as the Wagner-Nelson or Loo-Riegelman analysis. See also Welling, “Pharmacokinetics: Processes and Mathematics” (ACS Monograph 185, Amer. Chem. Soc., Washington, D.C., 1986). Treatment of the data in this manner yields an apparent in vivo drug release profile.

### Drug-Containing Composition

For the tri-layer, concentric core, and granular core embodiments of the present invention, the drug-containing composition 14 includes at least one drug and preferably additional excipients (the homogeneous core embodiment is discussed below). The drug-containing composition occupies a separate, substantially distinct region from the water-swellable composition. For the granular core embodiment, a substantially distinct region means that the water-swellable composition is present in a plurality of separate granules distributed throughout the drug-containing composition. When it is desired to deliver a relatively large dose of drug (about 100 mg or more) in a single dosage form, the drug-containing composition preferably comprises greater than about 50 wt % of the core. When it is desirable to deliver even greater amounts of drug (e.g., 150 mg or more), the drug-containing composition comprises preferably greater than about 60 wt % of the core, and more preferably greater than about 70 wt % of the core. Preferably, the drug-containing composition 14 is in contact with or in close proximity to the coating 18 which surrounds the dosage form.

The drug-containing composition(s) may contain one or more drugs, and in the case of the tri-layer dosage form, the first drug-containing composition 14a may contain a different drug than the second drug-containing composition 14b. The drug may be virtually any beneficial therapeutic agent and may comprise from 0.1 to 65 wt % of the drug-containing composition 14. In cases where the dose to be delivered is high (e.g., greater than about 100 mg), it is preferred that the drug comprise at least 35 wt % of the drug-containing composition 14. The drug may be in any form, either crystalline or amorphous. The drug may also be in the form of a solid dispersion.

The invention finds particular utility when the drug is a “low-solubility drug,” meaning that the drug is either “substantially water-insoluble” (which means that the drug has a minimum aqueous solubility at physiologically relevant pH (e.g., pH 1-8) of less than 0.01 mg/mL), or “sparingly water soluble,” that is, has a minimum aqueous solubility at physiologically relevant pH up to about 1 to 2 mg/mL, or has even low to moderate aqueous solubility, having a minimum aqueous solubility at physiologically relevant pH as high as about 10 to 20 mg/mL. In general, it may be said that the drug has a dose-to-aqueous solubility ratio greater than 10 mL, and more typically greater than 100 mL, where the drug solubility is the minimum value in mg/mL observed in any physiologically relevant aqueous solution (e.g., those with pH values between 1 and 8) including USP simulated gastric and intestinal buffers and the dose is in mg. The drug may be employed in its neutral (e.g., free acid, free base, or zwitterion) form, or in the form of its pharmaceutically acceptable salts as well as in anhydrous, hydrated, or solvated forms, and pro drugs.

Preferred classes of drugs include, but are not limited to, antihypertensives, antidepressants, antianxiety agents, anticlotting agents, anticonvulsants, blood glucose-lowering agents, decongestants, antihistamines, antitussives, anti-inflammatories, antipsychotic agents, cognitive enhancers, cholesterol-reducing agents, antiobesity agents, autoimmune disorder agents, anti-impotence agents, antibacterial and antifungal agents, hypnotic agents, anti-Parkinsonism agents, antibiotics, antiviral agents, anti-neoplastics, barbituates, sedatives, nutritional agents, beta blockers, emetics, anti-emetics, diuretics, anticoagulants, cardiotonics, androgens, corticoids, anabolic agents, growth hormone secretagogues, anti-infective agents, coronary vasodilators, carbonic anhydrase inhibitors, antiprotozoals, gastrointestinal agents, serotonin antagonists, anesthetics, hypoglycemic agents, dopaminergic agents, anti-Alzheimer's Disease agents, anti-ulcer agents, platelet inhibitors and glycogen phosphorylase inhibitors.

Specific examples of the above and other classes of drugs and therapeutic agents deliverable by the invention are set forth below, by way of example only. Specific examples of antihypertensives include prazosin, nifedipine, trimazosin, amlodipine, and doxazosin mesylate; a specific example of an antianxiety agent is hydroxyzine; a specific example of a blood glucose lowering agent is glipizide; a specific example of an anti-impotence agent is sildenafil citrate; specific examples of anti-neoplastics include chlorambucil, lomustine and echinomycin; specific examples of anti-inflammatory agents include betamethasone, prednisolone, piroxicam, aspirin, flurbiprofen and (+)-N-{4-[3-(4-fluorophenoxy)phenoxy]-2-cyclopenten-1-yl}-N-hyroxyurea; a specific example of a barbituate is phenobarbital; specific examples of antivirals include acyclovir, nelfinavir, and virazole; specific examples of vitamins/nutritional agents include retinol and vitamin E; specific examples of a blocker include timolol and nadolol; a specific example of an emetic is apomorphine; specific examples of a diuretic include chlorthalidone and spironolactone; a specific example of an anticoagulant is dicumarol; specific examples of cardiotonics include digoxin and digitoxin; specific examples of an androgen include 17-methyltestosterone and testosterone; a specific example of a mineral corticoid is desoxycorticosterone; a specific example of a steroidal hypnotic/anesthetic is alfaxalone; specific examples of an anabolic agent include fluoxymesterone and methanstenolone; specific examples of antidepression agents include fluoxetine, pyroxidine, venlafaxine, s rtraline, paroxetine, sulpiride, [3,6-dimethyl-2-(2,4,6-trimethyl-phenoxy)-pyridin-4-yl]-(lethylpropyl)-amine and 3,5-dimethyl-4-(3′-pentoxy)-2-(2′,4′,6′-trimethylphenoxy)pyridine; specific examples of an antibiotic include ampicillin and penicillin G; specific examples of an anti-infective include benzalkonium chloride and chlorhexidine; specific examples of a coronary vasodilator include nitroglycerin and mioflazine; a specific example of a hypnotic is etomidate; specific examples of a carbonic anhydrase inhibitor include acetazolamide and chlorzolamide; specific examples of an antifungal include econazole, terconazole, fluconazole, voriconazole and griseofulvin; a specific example of an antiprotozoal is metronidazole; a specific example of an imidazole-type anti-neoplastic is tubulazole; specific examples of an anthelmintic agent include thiabendazole and oxfendazole; specific examples of an antihistamine include astemizole, levocabastine, cetirizine, and cinnarizine; a specific example of a decongestant is pseudoephedrine; specific examples of antipsychotics include fluspirilene, penfluridole, risperidone and ziprasidone; specific examples of a gastrointestinal agent include loperamide and cisapride; specific examples of a serotonin antagonist include ketanserin and mianserin; a specific example of an anesthetic is lidocaine; a specific example of a hypoglycemic agent is acetohexamide; a specific example of an anti-emetic is dimenhydrinate; a specific example of an antibacterial is cotrimoxazole; a specific example of a dopaminergic agent is L-DOPA; specific examples of anti-Alzheimer agents are THA and donepezil; a specific example of an anti-ulcer agent/H2 antagonist is famotidine; specific examples of a sedative/hypnotic include chlordiazepoxide and triazolam; a specific example of a vasodilator is alprostadil; a specific example of a platelet inhibitor is prostacyclin; specific examples of an ACE inhibitor/antihypertensive include enalaprilic acid and lisinopril; specific examples of a tetracycline antibiotic include oxytetracycline and minocycline; specific examples of a macrolide antibiotic include azithromycin, clarithromycin, erythromycin and spiramycin; specific examples of glycogen phosphorylase inhibitors include [R-(R*S*)]-5-chloro-N-[2-hydroxy-3{methoxymethylamino}-3-oxo-1-(phenylmethyl)-propyl]-1H-indole-2-carboxamide and 5-chloro-1-Hindole-2-carboxylic acid [(1S)-benzyl(2R)-hydroxy-3-((3R,4S)dihydroxy-pyrrolidin-1-yl-)-oxypropyl]amide.

Further examples of drugs deliverable by the invention are the glucose-lowering drug chlorpropamide, the anti-fungal fluconazole, the antihypercholesterolemic atorvastatin calcium, the antipsychotic thiothixene hydrochloride, the anxiolytics hydroxyzine hydrochloride and doxepin hydrochloride, the anti-hypertensive amlodipine besylate, the antinflammatories piroxicam and celicoxib and valdicoxib, and the antibiotics carbenicillin indanyl sodium, bacampicillin-hydrochloride, troleandomycin, and doxycycline hyclate.

In an alternative embodiment, the drug is present in the form of a solid, amorphous dispersion. By solid, amorphous dispersion is meant that the drug is dispersed in a polymer so that a major portion of the drug is in a substantially amorphous or non-crystalline state, and its non-crystalline nature is demonstrable by x-ray diffraction analysis or by differential scanning calorimetry. The dispersion may contain from about 5 to 90 wt % drug. The polymer is aqueous-soluble and inert, and, when enhancement of bioavailability is desirable, is preferably concentration-enhancing. Suitable polymers and methods for making solid amorphous dispersions are disclosed in commonly assigned provisional patent applications Serial Nos. 60/119,406 and 60/119,400, the relevant disclosures of which are incorporated by reference. Suitable dispersion polymers include ionizable and non-ionizable cellulosic polymers, such as cellulose esters, cellulose ethers, and cellulose esters/ethers; and vinyl polymers and copolymers having substituents selected from the group consisting of hydroxyl, alkylacyloxy, and cyclicamido, such as polyvinyl pyrrolidone, polyvinyl alcohol, copolymers of polyvinyl pyrrolidone and polyvinyl acetate. Particularly preferred polymers include hydroxypropylmethyl cellulose acetate succinate (HPMCAS), hydroxypropyl methyl cellulose (HPMC), hydroxypropyl methyl cellulose phthalate (HPMCP), cellulose acetate phthalate (CAP), cellulose acetate trimellitate (CAT), and polyvinyl pyrrolidone (PVP). Most preferred are HPMCAS, HPMCP, CAP and CAT.

When the drug has a low solubility (less than about 20 mg/ml) it is preferable that the drug-containing composition also comprise an entraining agent. The use of an entraining agent is necessitated by the low-solubility drug, which due to its low-solubility does not dissolve sufficiently within the core 12 to be extruded in the absence of an entraining agent. The entraining agent suspends or entrains the drug so as to aid in the delivery of the drug through the delivery port(s) 20 to the environment of use. While not wishing to be bound by any particular theory, it is believed that upon imbibing water into the dosage form, the entraining agent imparts sufficient viscosity to the drug-containing composition to allow it to suspend or entrain the drug, while at the same time remaining sufficiently fluid to allow the entraining agent to pass through the delivery port(s) 20 along with the drug. It has been found that there is a good correlation between the usefulness of a material as an entraining agent and the viscosity of an aqueous solution of the material. The entraining agent generally is a material that has high water solubility and in operation forms aqueous solutions with viscosities of at least 50 centipoise (cp) and preferably aqueous solutions with viscosities of 200 cp or greater.

The amount of the entraining agent present in the drug-containing composition may range from about 5 wt % to about 98 wt %h of the drug-containing composition, preferably 10 wt % to 50 Wt % more preferably 10 wt % to 40 wt %. The entraining agent may be a single material or a mixture of materials. Examples of such materials include polyols, and oligomers of polyethers, such as ethylene glycol oligomers or propylene glycol oligomers. In addition, mixtures of polyfunctional organic acids and cationic materials such as amino acids or multivalent salts, such as calcium salts may be used. Of particular utility are polymers such as polyethylene oxide (PEO), polyvinyl alcohol, PVP, cellulosics such as hydroxyethyl cellulose (HEC), hydroxypropylcellulose (HPC), HPMC, methyl cellulose (MC), carboxy methyl cellulose (CMC), carboxyethylcellulose (CEC), gelatin, xanthan gum or any other water-soluble polymer that forms an aqueous solution with a viscosity similar to that of the polymers listed above. An especially preferred entraining agent is non-crosslinked PEO or mixtures of PEO with the other materials listed above.

When the drug and a polymeric entraining agent make up about 80 wt % or more of the drug-containing composition, then the entraining agent should have a sufficiently low molecular weight that ft becomes sufficiently fluid so that both the drug and entraining agent can be rapidly extruded from the dosage form, instead of swelling and rupturing the water-permeable coating that surrounds the dosage form. Thus, for example, when PEO is the drug-entraining agent, it is generally preferred that it have a molecular weight of from about 100,000 to about 300,000 daltons. (References to molecular weights of polymers herein and in the claims are to average molecular weights.)

When the drug and the entraining agent make up less than about 80 wt % of the drug-containing composition, a smaller portion of a more viscous entraining agent is preferred. For example, when the entraining agent is PEO, a lower fraction of a higher molecular weight of PEO from about 500,000 to 800,000 daltons may be used. Thus, there is an inverse relationship between the preferred PEO molecular weight and the weight fraction of the drug-containing composition that is drug and entraining agent. Thus, as the weight fraction decreases from about 0.9 to about 0.8, to about 0.7, to about 0.6, the preferred PEO molecular weight increases from about 200,000 daltons to about 400,000 daltons, to about 600,000 daltons, to about 800,000 daltons, respectively, and the weight fraction of entraining agent correspondingly decreases (the weight fraction of drug being relatively constant). It should be noted that for a particular formulation, the optimum PEO molecular weight for the entraining agent may vary higher or lower than those values by 20% to 50%. Likewise, when selecting an appropriate molecular weight of other polymeric entraining agents such as HEC, HPC, HPMC, or MC, as the weight fraction of entraining agent in the drug-containing composition is reduced, a higher molecular weight for the entraining agent is generally preferred.

In one embodiment of the invention, the drug-containing composition further comprises a swelling agent. The swelling agent is generally a water-swellable polymer that substantially expands in the presence of water. Inclusion of even a small amount of such a swellable polymer can significantly enhance the onset, rate, and completeness of drug delivery. The degree of swelling of a swelling agent can be assessed by compressing particles of the swelling agent in a press to form a compact of the material having a “strength” ranging from 3 to 16 Kp/cm², where strength is the hardness of the compact in Kp as measured with a Schleuniger Tablet Hardness Tester, model 6D, divided by its maximum cross-sectional area normal to the direction of force in cm². For example, about 500 mg of a swelling agent can be compressed in a 13/32-inch die using an “f press.” The swelling of a compact is measured by placing it between two porous glass frits in a glass cylinder and contacting it with a physiologically relevant test medium, such as simulated gastric or intestinal buffer, or water. The volume of the water-swollen compact after 16 to 24 hours contact with the test medium divided by its initial volume is termed the “swelling ratio” of the swelling agent. Generally, swelling agents suitable for inclusion in the drug layer are those water-swellable polymers that have swelling ratios, when water is the test medium, of at least 3.5, preferably greater than 5.

A preferred class of swelling agents comprises ionic polymers. Ionic polymers are generally polymers that have a significant number of functional groups that are substantially ionized in an aqueous solution over at least a portion of the physiologically relevant pH range 1 to 8. Such ionizable functional groups include carboxylic acids and their salts, sulfonic acids and their salts, amines and their salts, and pyridine salts. To be considered an ionic polymer, the polymer should have at least 0.5 milli-equivalents of ionizable functional groups per gram of polymer. Such ionic polymer swelling agents include sodium starch glycolate, sold under the trade name EXPLOTAB, and croscarmellose sodium, sold under the trade name AC-DISOL.

In one embodiment of the invention in which the drug-containing composition comprises a drug, a drug-entraining agent, and a swelling agent, the swelling agent is present in an amount ranging from about 2 to about 20 wt % of the drug-containing composition 14. In other embodiments of the invention, the swelling agent is optionally present in an amount ranging from 0 to about 20 wt %.

In another embodiment of the present invention, the drug-containing composition further comprises a fluidizing agent. As used herein, a “fluidizing agent” is a water-soluble compound that allows the drug-containing composition to rapidly b come fluid upon imbibing water when the dosage form is introduced into a us environment. Rapid fluidization of the drug-containing composition allows the composition to be extruded from the dosage form without a build-up of excessive pressure. This results in a relatively short time lag. That is, the time between introduction of the dosage form into the environment of use and the onset of drug delivery is relatively short. In addition, the inclusion of a fluidizing agent reduces the pressure within the core and thus reduces the risk of failure of the coating that surrounds the core of the dosage form. This is particularly important when a relatively rapid rate of drug release is desired, necessitating the use of a highly water-permeable coating that conventionally is relatively thin and weak. (By a rapid rate of release is generally meant that greater than 70 wt % of the drug originally present in the dosage form is released within 12 hours of the time the dosage form is introduced into the use environment.)

The fluidizing agent can be essentially any water-soluble compound that rapidly increases the fluidity of the drug-containing composition when water is imbibed into the core. Such compounds generally have aqueous solubilities of at least 30 mg/mL and generally have a relatively low molecular weight (less than about 10,000 daltons) such that upon imbibing a given quantity of water, the drug-containing composition rapidly becomes more fluid relative to a similar drug-containing composition that does not include the fluidizing agent. By more fluid is meant that the pressure required to extrude the drug through the delivery port(s) is lower than a similar composition without the fluidizing agent. This increased fluidity can be temporary, meaning that the increased fluidity occurs for only a short time after introduction of the dosage form to a use environment (e.g., 2 hours), or the increased fluidity can occur over the entire time the dosage form is in the use environment. Exemplary fluidizing agents are sugars, organic acids, amino acids, polyols, salts, and low-molecular weight oligomers of water-soluble polymers. Exemplary sugars are glucose, sucrose, xylitol, fructose, lactose, mannitol, sorbitol, maltitol, and the like. Exemplary organic acids are citric acid, lactic acid, ascorbic acid, tartaric acid, malic acid, fumaric, and succinic acid. Exemplary amino acids are alanine and glycine. Exemplary polyols are propylene glycol and sorbitol. Exemplary oligomers of low-molecular weight polymers are polyethylene glycols with molecular weights of 10,000 daltons or less. Particularly preferred fluidizing agents are sugars and organic acids. Such fluidizing agents are preferred as they often improve tableting and compression properties of the drug-containing composition relative to other fluidizing agents such as inorganic salts or low-molecular weight polymers.

In order for the fluidizing agent to rapidly increase the fluidity of the drug-containing composition at low water levels in the core 12 of the dosage form, th fluidizing agent must generally be present in an amount such that it makes up at least about 10 wt % of the drug-containing composition 14. To ensure that the drug-containing composition 14 does not become so fluid such that the drug-entraining agent cannot properly entrain or suspend the drug, particularly long after (12 hours or longer) introduction of the dosage form into the use environment, the amount of fluidizing agent generally should not exceed about 60 wt % of the drug-containing composition. In addition, as mentioned above, when a fluidizing agent is included, a drug-entraining agent with a higher molecular weight and correspondingly higher viscosity is generally included in the drug-containing composition, but at a lower level. Thus, for example, when the drug-containing composition comprises about 20 to 30 wt % of the low-solubility drug and about 30 wt % of a fluidizing agent such as a sugar, about 20 to 50 wt % of a high molecular weight polymer such as PEO with a molecular weight of about 500,000 to 800,000 daltons is preferable to a lower molecular weight PEO.

The drug-containing composition 14 may further include solubilizing agents that promote the aqueous solubility of the drug, present in an amount ranging from about 0 to about 30 wt % of the drug-containing composition 14. Examples of suitable solubilizing agents include surfactants; pH control agents such as buffers, organic acids and organic acid salts and organic and inorganic bases; glycerides; partial glycerides; glyceride derivatives; polyhydric alcohol esters; PEG and PPG esters; polyoxyethylene and polyoxypropylene ethers and their copolymers; sorbitan esters; polyoxyethylene sorbitan esters; carbonate salts; and cyclodextrins.

There are a variety of factors to consider when choosing an appropriate solubilizing agent for a drug. The solubilizing agent should not interact adversely with the drug. In addition, the solubilizing agent should be highly efficient, requiring minimal amounts to effect the improved solubility. It is also desired that the solubilizing agent have a high solubility in the use environment. For acidic, basic, and zwitterionic drugs, organic acids, organic acid salts, and organic and inorganic bases and base salts are known to be useful solubilizing agents. It is desired that these compounds have a high number of equivalents of acid or base per gram. The selection of solubilizing agent will therefore be highly dependent on the properties of the drug.

A preferred class of solubilizing agents for basic drugs is organic acids. Since basic drugs are solubilized by protonation, and since the solubility of basic drugs in an aqueous environment of pH 5 or higher is reduced and often may reach an extremely low value by pH 7.5 (as in the colon), it is believed that addition of an organic acid to the dosage form for delivery to the use environment with such drugs assists in solubilization and hence absorption of the drug. An exemplary basic drug is s rtraline, which has moderate solubility at low pH, low solubility at pH valu s abov 5 and extremely low solubility at pH of about 7.5. Even a slight decrease in the pH of the aqueous solution at high pH may result in dramatic increases in the solubility of basic drugs. In addition to simply lowering the pH, the presence of organic acids and their conjugate bases also raises the solubility at a given pH if the conjugate base salt of the basic drug has a higher solubility than the neutral form or the chloride salt of the drug.

It has been found that a preferred subset of organic acids meeting such criteria consists of citric, succinic, fumaric, adipic, malic and tartaric acids. The table below gives properties of these organic acids. Of these, fumaric and succinic are especially preferred when a high ratio of equivalents of acid per gram is desired. In addition, citric, malic, and tartaric acid have the advantage of extremely high water solubility. Succinic acid offers a combination of both moderate solubility and a high acid equivalent per gram value. Thus, the use of a highly soluble organic acid serves multiple purposes: it improves the solubility of the basic drug, particularly when the use environment is at a pH above about 5 to 6; it makes the drug-containing composition more hydrophilic so that it readily wets; and it dissolves, lowering the viscosity of the layer rapidly, thus acting as a fluidizing agent. Thus, by accomplishing multiple functions with a single ingredient, additional space is available for the low-solubility drug within the drug-containing composition.

### Properties of Organic Acid Solubilizing Agents

| | Equivalents | Water |
|---|---|---|
| Organic | Value | Solubility |
| Acid | (mEq/g) | (mg/mL) |
| Fumaric | 17.2 | 11 |
| Succinic | 16.9 | 110 |
| Citric | 15.6 | >2000 |
| Malic | 14.9 | 1750 |
| Adipic | 13.7 | 45 |
| Tartaric | 13.3 | 1560 |

For acidic drugs, solubility is increased as pH increases. Exemplary classes of solubilizing agents for acidic drugs include alkalinizing or buffering agents and organic bases. It is believed that addition of an alkylating agent or organic base to the dosage form assists in solubilization and hence absorption of the drug. Examples of alkylating or buff ring agents include potassium citrate, sodium bicarbonate, sodium citrate, dibasic sodium phosphate, and monobasic sodium phosphate. Examples of organic bases include meglumine, eglumine, monoethanol amine, diethanol amine, and triethanol amine.

The drug-containing composition 14 may optionally include a concentration-enhancing polymer that enhances the concentration of the drug in a use environment relative to control compositions that are free from the concentration-enhancing polymer. The concentration-enhancing polymer should be inert, in the sense that it does not chemically react with the drug in an adverse manner, and should have at least some solubility in aqueous solution at physiologically relevant pHs (e.g. 1-8). Almost any neutral or ionizable polymer that has an aqueous solubility of at least 0.1 mg/mL over at least a portion of the pH range of 1-8 may be suitable. Especially useful polymers are those discussed above for forming solid-amorphous dispersions of the drug with a polymer. Preferred polymers include HPMCAS, HPMC, HPMCP, CAP, CAT, and PVP. More preferred polymers included HPMCAS, HPMCP, CAP and CAT. Without being bound by any particular theory or mechanism of action, it is believed that the concentration-enhancing polymer prevents or retards the rate at which a drug, delivered from the dosage form and present in the use environment at a concentration greater than its equilibrium value, approaches its equilibrium concentration. Thus, when the dosage form is compared to a control dosage form that is identical except for the absence of the concentration-enhancing polymer, the concentration-enhancing polymer-containing dosage form provides, at least for a short time period, a greater concentration of dissolved drug in the use environment. Appropriate drug forms and concentration-enhancing polymers are discussed in commonly assigned pending patent application “Pharmaceutical Compositions Providing Enhanced Drug Concentrations” filed Dec. 23, 1999, U.S. provisional patent application No. 60/171,841, the relevant portions of which are herein incorporated by reference.

The drug-containing composition 14 may optionally include excipients that promote drug stability. Examples of such stability agents include pH control agents such as buffers, organic acids and organic acid salts and organic and inorganic bases and base salts. These excipients can be the same materials listed above for use as solubility-enhancing agents or fluidizing agents. Another class of stability agents is antioxidants, such as butylated hydroxy toluene (BHT), butylated hydroxyanisole (BHA), vitamin E, and ascorbyl palmitate. The amount of stability agent used in the drug-containing composition should be sufficient to stabilize the low-solubility drug. For pH control agents such as organic acids, the stability agent, when present, may range from 0.1 to 20 wt % of the drug-containing composition. Note that in some formulations, antioxidants such as BHT can lead to discoloration of the dosage form. In these cases, the amount of antioxidant used should be minimized so as to prevent discoloration. The amount of antioxidant used in the drug-containing composition generally ranges from 0 to 1 wt % of the drug-containing composition.

Finally, the drug-containing composition 14 may also include other conventional excipients, such as those that promote performance, tableting or processing of the dosage form. Such excipients include tableting aids, surfactants, water-soluble polymers, pH modifiers, fillers, binders, pigments, osmagents, disintegrants and lubricants. Exemplary excipients include microcrystalline cellulose; metallic salts of acids such as aluminum stearate, calcium stearate, magnesium stearate, sodium stearate, and zinc stearate; fatty acids, hydrocarbons and fatty alcohols such as stearic acid, palmitic acid, liquid paraffin, stearyl alcohol, and palmitol; fatty acid esters such as glyceryl (mono- and di-) stearates, triglycerides, glyceryl (palmitic stearic) ester, sorbitan monostearate, saccharose monostearate, saccharose monopalmitate, and sodium stearyl fumarate; alkyl sulfates such as sodium lauryl sulfate and magnesium lauryl sulfate; polymers such as polyethylene glycols, polyoxyethylene glycols, and polytetrafluoroethylene; and inorganic materials such as talc and dicalcium phosphate. In a preferred embodiment, the drug-containing composition 14 contains a lubricant such as magnesium stearate.

### Water-Swellable Composition

Referring again to FIGS. 1-3, the tri-layer, concentric core, and granular core dosage forms further comprise a water-swellable composition 16. The water-swellable composition greatly expands as it imbibes water through the coating 18 from the use environment. As it expands, the water-swellable composition increases the pressure within the core 12, causing extrusion of the fluidized drug-containing composition through the port(s) 20 into the environment of use. To maximize the amount of drug present in the dosage form and to ensure that the maximum amount of drug is released from the dosage form so as to minimize residual drug, the water-swellable composition should have a swelling ratio of at least about 2, preferably 3.5, and more preferably 5.

The water-swellable composition 16 comprises a swelling agent in an amount ranging from about 30 to 100 wt % of the water-swellable composition 16. The swelling agent is gen rally a water-swellable polymer that greatly expands in the presence of water. As discussed above in connection with the swelling agent of the drug-containing composition, the degree of swelling of a swelling agent, or the water-swellable composition itself, can be assessed by measuring its swelling ratio.

Suitable swelling agents for the water-swellable composition are generally hydrophilic polymers that have swelling ratios of about 2.0 or greater. Exemplary hydrophilic polymers include polyoxomers such as PEO, cellulosics such as HPMC and HEC, and ionic polymers. In general, the molecular weight of water swellable polymers chosen for the swelling agent is higher than that of similar polymers used as entraining agents such that, at a given time during drug release, the water-swellable composition 16 after imbibing water tends to be more viscous, less fluid, and more elastic relative to the drug-containing composition 14. In some cases the swelling agent may be even substantially or almost entirely water insoluble such that when partially water swollen during operation, it may constitute a mass of water-swollen elastic particles. Generally, the swelling agent is chosen such that, during operation, the water-swellable composition 16 generally does not substantially intermix with the drug-containing composition 14, at least prior to extruding a majority of the drug-containing composition 14. Thus, for example, when PEO is the swelling agent used in the water-swellable composition 16, a molecular weight of about 800,000 daltons or more is preferred and more preferably a molecular weight of 3,000,000 to 8,000,000 daltons.

A preferred class of swelling agents is ionic polymers, described above for use in various embodiments of the drug-containing composition 14. Exemplary ionic polymer swelling agents include sodium starch glycolate, sold under the trade name EXPLOTAB, croscarmellose sodium, sold under the trade name AC-DI-SOL, polyacrylic acid, sold under the trade name CARBOBOL, and sodium alginate sold under the trade name KELTONE.

The water-swellable composition may optionally further comprise osmotically effective agents, often referred to as “osmogens” or “osmagents.” The amount of osmagent present in the water-swellable composition may range from about 0 to about 40 wt % of the water-swellable composition. Typical classes of suitable osmagents are water-soluble salts and sugars that are capable of imbibing water to thereby effect an osmotic pressure gradient across the barrier of the surrounding coating. The osmotic pressure of a material can be calculated using the van't Hoff equation. (See, e.g., Thermodynamics, by Lewis and Randall). By “osmotically effective agent” is meant the inclusion of a material with low enough molecular weight, high enough solubility, and sufficient mass in the water-swellable composition that upon imbibing water from the use environment it forms an aqueous solution within the interior of the tablet such that its osmotic pressure exceeds that of the use environment, thereby providing an osmotic pressure driving force for permeation of water from the use environment into the tablet core. Typical useful osmagents include magnesium sulfate, magnesium chloride, calcium chloride, sodium chloride, lithium chloride, potassium sulfate, sodium carbonate, sodium sulfite, lithium sulfate, potassium chloride, sodium sulfate, d-mannitol, urea, sorbitol, inositol, raffinose, sucrose, glucose, fructose, lactose, and mixtures thereof.

In one embodiment of the invention, the water-swellable composition 16 is substantially free from an osmotically effective agent, meaning that there is either a sufficiently small amount of osmagent or that any osmagent present has sufficiently low solubility so as not to increase the osmotic pressure of the water-swellable composition 16 substantially beyond that of the use environment. In order for the dosage form to provide satisfactory release of drug in the absence of an osmagent in the water-swellable composition 16, and when the water-swellable polymer is not an ionic polymer, the dosage form should have a coating that is highly permeable to water. Such high-permeability coatings are described below. When the water-swellable composition 16 is substantially free of an osmotically effective agent, the water swellable composition preferably contains a substantial quantity, typically at least 10 wt % and preferably at least 50 wt %, of a highly swelling polymer such as sodium starch glycolate or sodium croscarmellose. As described earlier, highly swelling materials can be identified by measuring the “swelling ratio” of the material formed into a compact using the method described previously. When the water-soluble composition is substantially free of an osmotically effective solute, it is preferred that the swelling polymer have a swelling ratio of at least 3.5, preferably at least 5. The dosage form should also have a high strength coating to prevent rupture when highly swelling materials are used. Such coatings are described below.

The release of a drug relatively quickly without the inclusion of an osmagent in the water-swellable composition is a surprising result, since conventional wisdom in the art has held that osmagents should be included in the water-swellable composition to achieve good performance. Circumventing the need for inclusion of an osmagent provides several advantages. One advantage is that the space and weight which would otherwise be occupied by osmagent may be devoted to drug, thus permitting an increase in the amount of drug within the dosage form. Alternatively, the overall size of the dosage form may be decreased. In addition, eliminating the osmagent simplifies the process for manufacture of the dosage form, since the water-swellable composition 16 may omit the step of including an osmagent.

In one embodiment of the invention, the water swellable composition 16 comprises a swelling agent and a tableting aid. The preferred swelling agents (e.g., those that are highly swelling) are difficult to compress toga hardness suitable for use in the dosage form. However, it has been found that adding a tableting aid to the water-swellable composition in the amount of 5 to 50 wt % of the water-swellable composition 16 results in a material that compresses to a hardness suitable for use in the dosage form. At the same time inclusion of a tableting aid can adversely affect the swelling ratio of the water-swellable composition 16. Thus, the quantity and type of tableting aid used must be carefully selected. In general, hydrophilic materials with good compression properties should be used. Exemplary tableting aids include sugars such as lactose, in particular spray-dried versions sold under the trade name FASTFLOW LACTOSE, or xylitol, polymers such as microcrystalline cellulose, HPC, MC or HPMC. Preferred tableting aids are microcrystalline cellulose, both standard grades sold under the trade name AVICEL and silicified versions sold under the trade name PROSOLV and HPC. The amount of tableting aid is chosen to be sufficiently high so that the core 12 compresses well yet sufficiently low so that the water-swellable composition 16 still has a swelling ratio of at least 2, preferably 3.5, more preferably greater than 5. Typically, the amount is at least 20 but less than 60 wt %.

It is further desired that the mixture of swelling agent and tableting aid result in a material that has a “strength” of at least 3 Kiloponds (Kp)/cm², and preferably at least 5 Kp/cm². Here, “strength” is the fracture force, also known as the core “hardness,” required to fracture a core 12 formed from the material, divided by the maximum cross-sectional area of the core 12 normal to that force. In this test, the fracture force is measured using a Schleuniger Tablet Hardness Tester, model 6D. Both the compressed water-swellable composition 16 and resulting core 12 should have a strength of at least 3 Kp/cm², and preferably at least 5 Kp/cm².

In a preferred embodiment, the water-swellable composition 16 comprises a mixture of swelling agents in addition to a tableting aid. For example, the swelling agent croscarmellose sodium can be compressed into a compact with higher strength than the swelling agent sodium starch glycolate. However, the swelling ratio of croscarmellose sodium is lower than that of sodium starch glycolate.

The water-swellable composition 16 may also include solubility-enhancing agents or excipients that promote stability, tableting or processing of the dosage form of the same types mentioned above in connection with the drug-containing composition. However, it is generally preferred that such excipients comprise a minor portion of the water-swellable composition 16. In one preferred embodiment, the water-swellable composition 16 contains a lubricant such as magnesium stearate.

### The Homogeneous Core

The preceding discussion of drug-containing composition 14 and water-swellable composition 16 applies to the tri-layer, concentric core, and granular core embodiments. However, for the homogeneous core, the drug-containing composition 15 contains both the drug and swelling materials. In general, the drug-containing composition will simply be a mixture of materials suitable for use in the drug-containing composition 14 and the water-swellable composition 16 of the other embodiment described above. Thus, at a minimum, the drug-containing composition 15 comprises at least a drug, an entraining agent, and a swelling agent. The drug-containing composition 15 may optionally include a fluidizing agent, a solubility-enhancing agent, a concentration-enhancing polymer, a stability promoting agent, and/or conventional excipients discussed above in connection with the drug-containing composition. Likewise, the drug-containing composition may optionally also include osmogens, and/or tableting aids as discussed above in connection with the water-swellable composition.

The amounts of the respective materials will in general fall within the ranges described above in the discussion of the drug-containing composition and the water-swellable composition. In particular, preferred compositions for the homogeneous core embodiment are those that contain from 2 to about 30% of a swelling agent that has a swelling ratio of at least about 2 and preferably at least about 3.5, and more preferably at least about 5. Preferred swelling agents are ionic polymers such as carboxymethyl cellulose, sodium starch glycolate, crosscarmelose sodium, polyacrylic acid and sodium alginate. In addition, preferred homogeneous core compositions will also contain an entraining agent such as HEC, HPC, HPMC, or PEO in an amount from about 5 to about 80% of the core contents. Preferably, in addition to the drug, swelling agent, and entraining agent, the core also contains a fluidizing agent.

The various novel combinations of these agents in the core of the homogeneous core embodiment yield numerous advantages, including more rapid onset and more complete release of drug, relative to homogeneous core dosage forms previously known.

### The Core

The core 12 may be any known tablet that can be formed by an extrusion or compression process and be subsequently coated and utilized for delivery of drug to a mammal. The tablet can generally range in size from about 1 mm to about 10 cm for its longest dimension. The maximum size of the tablet will be different for different mammalian species. It can have essentially any shape such that its aspect ratio, defined as the tablet's longest dimension divided by the tablet's shortest dimension, ranges from about 1 to about 5. In addition, the dosage form may comprise two or more relatively small tablets contained in a relatively large container such as a capsule.

Exemplary core 12 shapes are spheres, ellipsoids, cylinders, capsule or caplet shapes and any other known shape. The core 12, following coating, can comprise the entire or a portion of the dosage form. The final dosage form can be for oral, rectal, vaginal, subcutaneous, or other known method of delivery into the environment of use. When the dosage form 10 is intended for oral administration to a human, the core 12 generally has an aspect ratio of about 3 or less, a longest dimension of about 2 cm or less and a total weight of about 1.5 g or less and preferably a total weight of about 1.0 g or less.

To form the dosage form, the ingredients comprising the drug-containing composition 14 and the water-swellable composition 16 are first mixed or blended using processes known in the art. See for example, Lachman, et al., “The Theory and Practice of Industrial Pharmacy” (Lea & Febiger, 1986). For example, a portion of the ingredients of the drug-containing composition 14 can first be blended, then wet granulated, dried, milled, and then blended with additional excipients prior to tableting. Similar processes can be used to form the water-swellable composition.

Once the materials are properly mixed, the core 12 is formed using procedures known in the art, such as compression or extrusion.

For tri-layer dosage forms, the method used to make the core depends on whether the two drug-containing compositions 14a and 14b are the same. Where they are the same, a single drug-containing composition is prepared. A portion of the drug-containing composition mixture is placed in a tablet press and leveled by lightly tamping with the press. The desired amount of water-swellable composition 16 is then added. A second portion of the drug-containing composition is then added on top of the water-swellable composition. The tablet is then compressed.

Wher the two drug-containing compositions 14a and 14b differ, then each drug-containing composition 14a and 14b are separately prepared. The tablet is prepared by placing first the drug-containing composition 14a in a tablet press and leveling by lightly tamping with the press. The desired amount of water-swellable composition 16 is then added. The desired amount of the drug-containing composition 14b is then added on top of the water-swellable composition 16. The tablet is then compressed.

For the concentric core dosage form, the core 12 is first prepared by placing the desired amount of the water-swellable composition 16 in a press and compressing to form a small initial core. A first portion of the drug-containing composition is placed in a larger press, gently leveled and lightly compressed. The small initial core of water-swellable composition 16 is then placed on top of the first portion of the drug-containing composition and centered. The remaining amount of the drug-containing composition 14 is then added to the press. The tablet is compressed to the desired hardness.

For the granular dosage form, the water-swellable composition 16 is prepared and formed into granules using any conventional method, such as wet or dry granulation. The granules may vary in size from very small particulates less than 0.1 mm in diameter to large particles (up to 2 mm) that are each a significant fraction of the total volume of the dosage form. A preferred size range is an average diameter of between 0.1 mm and 2 mm, and more preferred is an average diameter of between 0.5 and 1.5 mm. In use, the size of the granules should be chosen so that upon swelling the granules are larger than the delivery ports in the coating. The granules will therefore be retained within the coating and displace the drug-containing composition, which is extruded through the delivery ports. The tablet core is prepared by adding the prepared granules of water-swellable composition 16 to the drug-containing composition 14, so that the granules are distributed throughout the drug-containing composition. The resulting composition is then placed into a tablet press, and then compressed.

Finally, for the homogeneous core dosage form, the drug-containing composition 15 is formed by mixing all of the ingredients using any conventional method to form a relatively homogeneous mixture. The mixture is then added to a tablet press, and then compressed. In contrast to the granular core embodiment, the swelling agent is present in particles having a small enough size (e.g., less than 0.1 mm) so that even when swollen the swelling agent particles are extruded through the delivery port along with the other ingredients in the core.

The amount of force used to compress the tablet core will depend on the size of the dosage form, as well as the compressibility and flow characteristics of the compositions. Typically, a pressure is used that results in a tablet with a strength of 3 to 20 Kp/cm².

### The Coating

Following formation of the core 12, coating 18 is applied. Coating 18 should have both a sufficiently high water permeability that the drug can be delivered within the desired time frame, and high strength, while at the same time be easily manufactured. A water permeability is chosen to control the rate at which water enters the core, thus controlling the rate at which drug is delivered to the use environment. Where a high dose of a low-solubility drug is required, the low solubility and high dose combine to make it necessary to use a high permeability coating to achieve the desired drug release profile while keeping the tablet acceptably small. High strength is required to ensure the coating does not burst when the core swells as it imbibes water, leading to an uncontrolled delivery of the core contents to the use environment. The coating must be easily applied to the dosage form with high reproducibility and yield. Furthermore, the coating must be non-dissolving and non-eroding during release of the drug-containing composition, generally meaning that it be sufficiently water-insoluble that drug is substantially entirely delivered through the delivery port(s) 20, in contrast to delivery via permeation through coating 18.

As described above, the coating 18 is highly water-permeable to allow rapid imbibition of water into core 12 and as a result a rapid release of the drug-containing composition 14. A relative measure of the water permeability of the coating can be made by conducting the following experiment. Finished dosage forms are placed in an open container which is in turn placed in an environmental chamber held at a constant temperature of 40 □C and a constant relative humidity of 75%. The initial rate of weight gain of the dry dosage forms, determined by plotting the weight of the dosage form versus time, divided by the surface area of the dosage form yields a value termed “water flux (40/75).” The water flux (40/75) for a dosage form has been found to be a useful relative measure of the water permeabilities of coatings. When a rapid release of the drug is desired, the coating should have a water flux (40/75) value of at least 1.0×10⁻³ gm/hr·cm², and preferably at least 1.3×10⁻³ gm/hr·cm².

As mentioned, the coating should also have a high strength to ensure the coating 18 does not burst when the core swells due to imbibition of water from the use environment. A relative measure of coating strength can be made by conducting the following experiment that measures the “durability” of the coating. Finished tablets are placed into an aqueous medium for 10 to 24 hours, allowing the core to imbibe water, swell, and release drug to the media. The swollen dosag form can then be tested in a hardness tester, such as a Model 6D Tablet Tester manufactured by Schleuniger Pharmatron, Inc. When the delivery port(s) located on the face(s) of the dosage form, the-dosage form is placed into the tester so that its delivery port(s) (20) faces one side of the compression plates such that the delivery port(s) is blocked by the compression plate. The force, in Kp, required to rupture the coating is then measured. The durability of the coating is then calculated by dividing the measured rupture force by the maximum cross-sectional area of the dosage form normal to the applied force. Preferably, the coating has a durability of at least 1 Kp/cm², more preferably at least 2 Kp/cm², and even more preferably at least 3 Kp/cm². Coatings with this or greater durability ensure virtually no burst tablets when the dosage forms are tested in vivo.

Coatings with these characteristics can be obtained using hydrophilic polymers such as plasticized and unplasticized cellulose esters, ethers, and ester-ethers. Particularly suitable polymers include cellulose acetate (“CA”), cellulose acetate butyrate, and ethyl cellulose. A particularly preferred set of polymers are cellulose acetates having acetyl contents of 25 to 42%. A preferred polymer is CA having an acetyl content of 39.8%, and specifically, CA 398-10 manufactured by Eastman of Kingsport, Tenn., having an average molecular weight of about 40,000 daltons. Another preferred CA having an acetyl content of 39.8% is high molecular weight CA having an average molecular weight greater than about 45,000, and specifically, CA 398-30 (Eastman) reported to have an average molecular weight of 50,000 daltons. The high molecular weight CA provides superior coating strength, which allows thinner coatings and thus higher permeability.

Coating is conducted in conventional fashion by first forming a coating solution and then coating by dipping, fluidized bed coating, or preferably by pan coating. To accomplish this, a coating solution is formed comprising the coating polymer and a solvent. Typical solvents useful with the cellulosic polymers noted above include acetone, methyl acetate, ethyl acetate, isopropyl acetate, n-butyl acetate, methyl isobutyl ketone, methyl propyl ketone, ethylene glycol monoethyl ether, ethylene glycol monoethyl acetate, methylene dichloride, ethylene dichloride, propylene dichloride, nitroethane, nitropropane, tetrachloroethane, 1,4-dioxane, tetrahydrofuran, diglyme, and mixtures thereof. A particularly preferred solvent is acetone. The coating solution typically will contain 3 to 15 wt % of the polymer, preferably 5 to 10 wt %, most preferably 7 to 10 wt %.

The coating solution may also comprise pore-formers, non-solvents, or plasticizers in any amount so long as the polymer remains substantially soluble at the conditions used to form the coating and so long as the coating remains water-permeable and has sufficient strength. Pore-formers and their use in fabricating coatings are described in U.S. Pat. Nos. 5,612,059 and 5,698,220, the pertinent disclosures of which are incorporated h rein. The term “pore former,” as used herein, refers to a material added to the coating solution that has low or no volatility relative to the solvent such that it remains as part of the coating following the coating process but that is sufficiently water swellable or wat r soluble such that, in the aqueous use environment it provides a water-filled or water-swollen channel or “pore” to allow the passage of water thereby enhancing the water permeability of the coating. Suitable pore-formers include polyethylene glycol (PEG), PVP, PEO, HEC, HPMC and other aqueous-soluble cellulosics, water-soluble acrylate or methacrylate esters, polyacrylic acid and various copolymers and mixtures of these water soluble or water swellable polymers. Enteric polymers such as cellulose acetate phthalate (CAP) and HPMCAS are included in this class of polymers. The pore former can also be a water soluble, pharmaceutically acceptable material, such as a sugar, organic acid, or salt. Examples of suitable sugars include sucrose and lactose; examples of organic acids include citric acid and succinic acid; examples of salts include sodium chloride and sodium acetate. Mixtures of such compounds may also be used. The pore former may be soluble in the solvent used in the coating solution, or it may be insoluble, such that the coating solution is a slurry or suspension. A particularly preferred pore former is PEG having an average molecular weight from 1000 to 8000 daltons. A particularly preferred PEG is one having a molecular weight of 3350 daltons. The inventors have found that to obtain a combination of high water permeability and high strength when PEG is used as a pore former, the weight ratio of CA:PEG should range from about 6.5:3.5 to about 9:1.

The addition of a non-solvent to the coating solution results in exceptional performance. By “non-solvent” is meant any material added to the coating solution that substantially dissolves in the coating solution and reduces the solubility of the coating polymer or polymers in the solvent. In general, the function of the non-solvent is to impart porosity to the resulting coating. As described below, porous coatings have higher water permeability than an equivalent weight of a coating of the same composition that is not porous and this porosity, when the pores are gas filled, as is typical when the non-solvent is volatile, is indicated by a reduction in the density of the coating (mass/volume). Although not wishing to be bound by any particular mechanism of pore formation, it is generally believed that addition of a non-solvent imparts porosity to the coating during evaporation of solvent by causing the coating solution to undergo liquid-liquid phase separation prior to solidification. As described below for the case of using water as the non-solvent in an acetone solution of cellulose acetate, the suitability and amount of a particular candidate material can be evaluated for use as a non-solvent by progressively adding the candidate non-solvent to the coating solution until it becomes cloudy. If this does not occur at any addition level up to about 50 wt % of the coating solution, it generally is not appropriate for use as a non-solvent. When clouding is observed, termed the “cloud point,” an appropriate level of non-solvent for maximum porosity is the amount just below the cloud point. When lower porosities are desired, the amount of non-solvent can be reduced as low as desired. It has been found that suitable coatings can be obtained when the concentration of non-solvent in the coating solution is greater than about 20% of the non-solvent concentration that results in the cloud point.

Suitable non-solvents are any materials that have appreciable solubility in the solvent and that lower the coating polymer solubility in the solvent. The preferred non-solvent depends on the solvent and the coating polymer chosen. In the case of using a volatile polar coating solvent such as acetone or methyl ethyl ketone, suitable non-solvents include water, glycerol, ethylene glycol and its low molecular-weight oligomers (e.g., less than about 1,000 daltons), propylene glycol and its low molecular weight oligomers (e.g., less than about 1,000 daltons), C₁ to C₄ alcohols such as methanol or ethanol, ethylacetate, acetonitrile and the like.

In general, to maximize its effect, (e.g., formation of pores), the non-solvent should have similar or less volatility than the coating solution solvent such that, during initial evaporation of the solvent during the coating process, sufficient non-solvent remains to cause phase separation to occur. In many cases, where a coating solution solvent such as acetone is used, water is a suitable non-solvent. For acetone solutions comprising 7 wt % CA and 3 wt % PEG, the cloud point at room temperature is at about 23 wt % water. Thus the porosity and in turn the water permeability (which increases with increasing porosity) can be controlled by varying the water concentration up to near the cloud point. For acetone solutions comprising CA and PEG with a total concentration of about 10 wt % it is desired that the coating solution contain at least 4 wt % water to obtain a suitable coating. When a higher porosity, and thus a higher water permeability is desired (to obtain a faster release rate), the coating solution should contain at least about 15 wt % water.

In one embodiment of the invention, the coating solution is homogeneous, in that when the polymer, solvent, and any pore formers or non-solvents are mixed, the solution comprises a single phase. Typically, a homogenous solution will be clear, and not be cloudy as discussed above.

When using CA 398-10, exemplary coating solution weight ratios of CA:PEG 3350:water are 7:3:5, 8:2:5, and 9:1:5, with the remainder of the solution comprising a solvent such as acetone. Thus, for example, in a solution having a w ight ratio of CA:PEG 3350:water of 7:3:5, CA comprises 7 wt %h of the solution, PEG 3350 comprises 3 wt % of the solution, water comprises 5 wt % of the solution, and acetone comprises the remaining 85 wt %. Preferred coatings are generally porous even in the dry state (prior to delivery to the aqueous use environment). By “porous” is meant that the coating has a dry-state density less than the density of the nonporous coating material. By “nonporous coating material” is meant a coating material formed by using a coating solution containing no non-solvent, or the minimum amount of non-solvent required to produce a homogeneous coating solution. The coating in the dry state has a density that is less than 0.9 times, and more preferably less than 0.75 times that of the nonporous coating material. The dry-state density of the coating can be calculated by dividing the coating weight (determined from the weight gain of the tablets before and after coating) by the coating volume (calculated by multiplying the coating thickness, as determined by optical or scanning electron microscopy, by the tablet surface area). The porous nature of the coating is one of the factors that leads to the combination of high water permeability and high strength of the coating.

The coatings may also be asymmetric, meaning that there is a gradient of density throughout the coating thickness. Generally, the outside surface of the coating will have a higher density than the coating nearest the core.

The coating can optionally include a plasticizer. A plasticizer generally swells the coating polymer such that the polymer's glass transition temperature is lowered, its flexibility and toughness increased and its permeability altered. When the plasticizer is hydrophilic, such as polyethylene glycol, the water permeability of the coating is generally increased. When the plasticizer is hydrophobic, such as diethyl phthalate or dibutyl sebacate, the water permeability of the coating is generally decreased.

It should be noted that additives can function in more than one way when added to the coating solution. For example, PEG can function as a plasticizer at low levels while at higher levels it can form a separate phase and act as a pore former. In addition, when a non-solvent is added, PEG can also facilitate pore formation by partitioning into the non-solvent-rich phase once liquid-liquid phase separation occurs.

The weight of the coating around the core depends on the composition and porosity of the coating, the surface to volume ratio of the dosage form, and the desired drug release rate, but generally should be present in an amount ranging from about 3 to 30 wt %, preferably from 8 to 25 wt %, based on the weight of the uncoated core. However, a coating weight of at least about 8 wt % is generally preferred so as to assure sufficient strength for reliable performance, and more preferably a coating greater than about 13 wt %.

While porous coatings based on CA, PEG, and water yield excellent results, other pharmaceutically acceptable materials may be used so long as the coating has the requisite combination of high water p rm ability, high strength; and ease of manufacture. Further, such coatings may be dense, or asymmetric, having one or more dense layers and one or more porous layers, as described in U.S. Pat. Nos. 5,612,059 and 5,698,220.

The coating 18 must also contain at least one delivery port 20 in communication with the interior and exterior of the coating to allow for release of the drug-containing composition to the exterior of the dosage form. The delivery port can range in size from about the size of the drug particles, and thus could be as small as 1 to 100 microns in diameter and may be termed pores, up to about 5000 microns in diameter. The shape of the port may be substantially circular, in the form of a slit, or other convenient shape to ease manufacturing and processing. The port(s) may be formed by post-coating mechanical or thermal means or with a beam of light (e.g., a laser), a beam of particles, or other high-energy source, may be formed by drilling completely through the dosage form, or may be formed in situ by rupture of a small portion of the coating. Such rupture may be controlled by intentionally incorporating a relatively small weak portion into the coating. Delivery ports may also be formed in situ by erosion of a plug of water-soluble material or by rupture of a thinner portion of the coating over an indentation in the core. Delivery ports may be formed by coating the core such that one or more small regions remains uncoated. In addition, the delivery port can be a large number of holes or pores that may be formed during coating, as in the case of asymmetric membrane coatings of the type disclosed in U.S. Pat. Nos. 5,612,059 and 5,698,220, the disclosures of which are incorporated by reference. When the delivery pathways are pores there can be a multitude of such pores that range in size from about 1 □m to greater than about 100 □m. During operation, one or more of such pores may enlarge under the influence of the hydrostatic pressure generated during operation. The number of delivery ports 20 may vary from 1 to 10 or more. In aggregate, the total surface area of core exposed by delivery ports is less than about 5%, and more typically less than about 1%.

At least one delivery port is formed through the coating so that the drug-containing composition will be extruded out of the delivery port by the swelling action of the water-swellable composition. For the tri-layer embodiment, it is desired to have at least one delivery port located on each of the respective faces of the tablet opposite each of the drug-containing compositions 14a and 14b. For the remaining embodiments, the location of the delivery ports is not critical, since any location will provide a delivery port in communication with either the drug-containing composition 14, in the case of the concentric core and granular core embodiments, or the drug-containing composition 15 in the case of the homogeneous core embodiment. Thus, for these embodiments the delivery port may be located at any location on the coating.

Other features and embodiments of the invention will become apparent from the following examples which are given for illustration of the invention rather than for limiting its intended scope.

### EXAMPLE 1

Exemplary dosage forms of the present invention were made with a tri-layer geometry of the type depicted in FIG. 1. The tri-layer core consisted of a drug containing composition distributed evenly between the top and bottom tablet layers and a water-swellable composition comprising the middle layer.

To form the drug-containing composition the following materials were wet granulated (see Table A): 35 wt % of the citrate salt of 1-[4-ethoxy-3-(6,7-dihydro-1-methyl-7-oxo-3-propyl-1H-pyrazolo[4,3-d]pyrimidin-5-yl)phenylsulphony]-4-methylpiperazine, also known as sildenafil citrate (hereinafter referred to as Drug 1) having a solubility of about 20 □g/mL at pH 6, 30 wt % xylitol (trade name XYLITAB 200), 29 wt % PEO with an average molecular weight of 600,000 daltons, 5 Wt % sodium starch glycolate (trade name EXPLOTAB), and 1 wt % magnesium stearate. The drug-containing composition ingredients were first combined with 26% of the total PEO, and without the magnesium stearate, in a twinshell mixer and blended for 10 minutes. Next, the ingredients were milled using a hammer mill and passed through a 0.065-inch screen. This material was blended again for 10 minutes in a twinshell mixer. An intensifier bar was inserted into the twinshell mixer and the material was granulated using deionized water. The granules were tray-dried in a 40 □C oven overnight, then milled the following morning using a hammer mill and passed through a 0.065-inch screen. The drug-containing composition ingredients were again placed in a twinshell mixer and the remaining 74% of the total PEO was added to the mixer. The drug-containing composition ingredients were blended for 10 minutes, the magnesium stearate was added, and the mixture was blended again for 4 minutes.

To form the water-swellable composition (see Table B), the following materials were blended: 74.5 wt % EXPLOTAB, 24.5 wt % of the tableting aid silicified microcrystalline cellulose (trade name PROSOLV 90), and 1.0 Wt % magnesium stearate. The water-swellable composition ingredients were first combined without the magnesium stearate in a twinshell mixer and blended for 20 minutes. An intensifier bar was ins rted into the twinshell mixer and the material was granulated using deionized water. The granules were tray-dried in a 40 □C oven overnight, then milled the following morning using a hammer mill and passed through a 0.065-inch scr en. The water-swellable composition ingredients were again placed in a twinshell mixer, the magnesium stearate was added, and the mixture was blended for 4 minutes.

Tablet cores were formed by placing 200 mg of drug-containing composition in a standard 13/32 inch die and gently leveling with the press. Then, 100 mg water-swellable composition was placed in the die on top of the drug-containing composition and leveled. The second half of the drug-containing composition (200 mg) was added and the tablet core compressed to a hardness of about 11 Kp. The resulting tri-layer tablet core had a total weight of 500 mg and contained a total of 28.3 wt % Drug 1 (141.5 mg), 24.3 wt % XYLITAB 200, 22.3 wt % PEO 600,000 daltons, 19.0 wt % EXPLOTAB, 4.9 wt % PROSOLV 90, and 1.2 wt % magnesium stearate.

Coatings were applied by a Vector LDCS-20 pan coater. The coating solution contained cellulose acetate (CA 398-10 from Eastman Fine Chemical, Kingsport, Tenn.), polyethylene glycol having a molecular weight of 3350 daltons (PEG 3350, Union Carbide), water, and acetone in a weight ratio of 7/3/5/85 (wt %). The flow rate of the inlet heated drying air of the pan coater was set at 40 ft³/min with the outlet temperature set at 25 □C. Nitrogen at 20 psi was used to atomize the coating solution from the spray nozzle, with a nozzle-to-bed distance of 2 inches. The pan rotation was set to 20 rpm. The so-coated tablets were dried at 50 □C in a convection oven. The final dry coating weight amounted to 47.5 mg or 9.5 wt % of the tablet core. Five 900 μm diameter holes were then laser-drilled in the coating on each drug-containing composition side of the tablet to provide 10 delivery ports per tablet. Table C summarizes the characteristics of the dosage form.

To simulate in vivo drug dissolution, tablets were placed in 900 mL of a simulated gastric solution (10 mM HCl, 100 mM NaCl, pH 2.0, 261 mOsm/kg) in a USP type 2 dissoette flask. Samples were taken at periodic intervals using a VanKel VK8000 autosampling dissoette with automatic receptor solution replacement. Tablets were placed in a wire support, the paddle height was adjusted, and the dissoette flasks were stirred at 100 rpm at 37 □C. The autosampler dissoette device was programmed to periodically remove a sample of the receptor solution, and the drug concentration was analyzed by HPLC using a Waters Symmetry C₁₈ column. The mobile phase consisted of 0.05 M triethanolamine (pH 3)/methanol/acetonitrile in a volume ratio of 58/25/17. Drug concentration was calculated by comparing UV absorbance at 290 nm to the absorbance of Drug 1 standards. Results are shown in Table 1 and summarized in Table F.

TABLE 1

| | Drug |
|---|---|
| Time (hours) | (wt % released) |
| 0 | 0 |
| 1 | 5 |
| 2 | 19 |
| 3 | 32 |
| 6 | 63 |
| 9 | 83 |
| 12 | 94 |
| 15 | 95 |
| 18 | 96 |
| 21 | 99 |
| 24 | 100 |

The data show that 19 wt % of the drug was released within 2 hours, 83 wt % within 9 hours, and 100 wt % of the drug was released within 24 hours. Thus, the present invention provided a rapid release of over 80 wt % within 9 hours and no residual value at 24 hours, of a relatively high dose (97 mgA) of a low-solubility drug in a relatively low mass (547.5 mg) dosage form.

### EXAMPLES 2A-2D

These examples demonstrate the inventive delivery of various drugs from tri-layer tablets. For the tablets of Example 2A, the drug-containing composition consisted of 28 wt % sertraline HCl (Drug 2) having a solubility of 0.2 mg/mL at pH 7, 37 wt % XYLITAB 200, 29 wt % PEO with an average molecular weight of 600,000 daltons, 5 wt % EXPLOTAB, and 1 wt % magnesium stearate. The drug-containing composition ingredients were first combined without the magnesium stearate and blended for 20 minutes in a TURBULA mixer. The ingredients were milled using a hammer mill and passed through a 0.065-inch screen, then blended again for 20 minutes in the TURBULA mixer. Next, magnesium stearate was added and the drug-containing composition was blended again for 4 minutes in the same mixer.

To form the water-swellable composition, the following materials were blended: 75.5 wt % EXPLOTAB, 25 wt % microcrystalline cellulose (AVICEL PH 102), and 2.5 wt % magnesium stearate. The water-swellable composition ingredients were first combin d without the magnesium stearate and blended for 20 minutes in a TURBULA mixer. Next, magnesium stearate was added and the water-swellable composition was blended again for 4 minutes in the same mixer.

Tablet cores were formed by placing 200 mg of drug-containing composition in a standard 13/32 inch die and gently leveling with the press. Then, 100 mg water-swellable composition was placed in the die on top of the drug-containing composition and leveled. The second half of the drug-containing composition (200 mg) was added and the tablet core compressed to a hardness of about 11 Kp. The resulting tri-layer tablet core had a total weight of 500 mg and contained a total of 22.5 wt % Drug 2 (112.5 mg), 29.5 wt % XYLITAB 200, 23 wt % PEO 600,000 daltons, 18.5 wt % EXPLOTAB, 5 wt % AVICEL, and 1.5 wt % magnesium stearate.

Coatings were applied as described in Example 1. The final dry coating weight amounted to 50.5 mg or 10.1 wt % of the tablet core. Five 900 μm diameter holes were then laser-drilled in the coating on each side of the tablet to provide 10 delivery ports per tablet. Table C summarizes the characteristics of the dosage form.

Dissolution tests were performed by placing the tablets in 900 mL of a simulated gastric solution (10 mM HCl, 100 mM NaCl, pH 2.0, 261 mOsm/kg) for 2 hours, then transferring the tablets to 900 mL of a simulated intestinal environment solution (6 mM KH₂PO₄, 64 mM KCl, 35 mM NaCl, pH 7.2, 210 mOsm/kg), both solutions being stirred at 100 rpm. A residual dissolution test was performed as described in the Detailed Description section. Residual drug was analyzed by HPLC using a Phenomenex Ultracarb 5 ODS 20 column. The mobile phase consisted of 35 vol % TEA-acetate buffer (3.48 mL triethanolamine and 2.86 mL glacial acetic acid in 1L HPLC H₂O) in acetonitrile. Drug concentration was calculated by comparing UV absorbance at 230 nm to the absorbance of sertraline standards. The amount of drug remaining in the tablets was subtracted from the total initial amount of drug in the tablet to obtain the amount released at each time interval. The results are presented in Table 2 and summarized in Table F.

For the tablets of Example 2B, the drug-containing composition consisted of 33 wt % of the mesylate salt of the drug 4-[3-[4-(2-methylimidazol-1-yl) phenylthio] phenyl]-3,4,5,6-tetrahydro-2H-pyran-4-carboxamide hemifumarate (Drug 3) having a solubility of 3.7 mgA/mL at pH 4, 31 wt % XYLITAB 200, 30 wt % PEO with an average molecular weight of 600,000 daltons, 5 wt % EXPLOTAB, and 1 wt % magnesium stearate (see Table A). The drug-containing composition ingredients were first combined without the magnesium stearate and blended for 20 minutes in a TURBULA mixer. The ingredients were milled using a hammer mill and passed through a 0.065-inch screen, then blended again for 20 minutes in the TURBULA mixer. Next, magnesium stearate was added and the drug-containing composition was blended again for 4 minutes in the same mixer.

The water-swellable composition consisted of 74.5 wt % EXPLOTAB, 24.5 wt % PROSOLV 90, and 1 wt % magnesium stearate. The water-swellable composition ingredients were first combined without the magnesium stearate in a twinshell mixer and blended for 20 minutes. An intensifier bar was inserted into the twinshell mixer and the material was granulated using deionized water. The granules were tray-dried in a 40 □C oven overnight, then milled the following morning using a hammer mill and passed through a 0.065-inch screen. The water-swellable composition ingredients were again placed in a twinshell mixer, the magnesium stearate was added, and the mixture was blended for 4 minutes.

Tablets for Example 2B were compressed and coated as described in Example 1. The resulting tri-layer tablet cores had a total weight of 500 mg and contained a total of 25.9 wt % Drug 3 (129.5 mg), 25.0 wt %/o XYLITAB 200, 23.9 wt % PEO 600,000 daltons, 19.1 wt % EXPLOTAB, 4.9 wt % PROSOLV 90, and 1.2 wt % magnesium stearate. The final dry coating weight amounted to 46.5 mg or 9.3 wt % of the tablet core. Five 900 μm diameter holes were then laser-drilled in the coating on each side of the tablet to provide 10 delivery ports per tablet.

Dissolution tests were performed on these tablets in accordance with the procedure described for Example 2A above, with the following exceptions: dissoette stir speed was 50 rpm, and residual drug was analyzed by dissolving tablets in 0.1 N HCl and measuring UV absorbance at 258 nm. Results are shown in Table 2 and summarized in Table F.

For the tablets of Example 2C, the drug-containing composition consisted of 35 wt % of nifedipine (Drug 4) having a solubility of 26 μg/mL in phosphate-buffered saline at pH 6.5, 30 wt % XYLITAB 200, 29 wt % PEO with an average molecular weight of 600,000 daltons, 5 wt % EXPLOTAB, and 1 Wt % magnesium stearate (see Table A). The drug-containing composition was processed as described in Examples 2A and 2B above.

The water-swellable composition consisted of 74.5 wt % EXPLOTAB, 25 wt % AVICEL PH200, and 0.5 wt % magnesium stearate. The water-swellable composition ingredients were first combined without the magnesium stearate and blended for 20 minutes in a TURBULA mixer. Next, magnesium stearate was added and the water-swellable composition was blended again for 4 minutes in the same mixer.

Tablets for Example 2C were compressed and coated as described in Example 1, with all weighing and tabletting procedures performed under low-light conditions (nifedipine is light-sensitive). The resulting tri-layer tablet cores had a total weight of 500 mg and contained a total of 28 wt % Drug 4 (140 mg), 24 wt % XYLITAB 200, 23 wt % PEO 600,000, 18.9 wt % EXPLOTAB, 5 wt % AVICEL, and 1.1 wt % magnesium stearate. The final dry coating weight amounted to 45.5 mg or 9.1 wt % of the tablet core. Five 900 μm diameter holes were then laser-drilled in the coating on each side of the tablet to provide 10 delivery ports per tablet.

Dissolution tests were performed on these tablets in accordance with the procedure described for Example 2A above, with the following exceptions: residual drug was analyzed by HPLC using a C₁₈ column with a mobile phase of 50% water/25% methanol/25% acetonitrile (vol. %) and UV detection at 235 nm. Results are shown in Table 2 and summarized in Table F.

For the tablets of Example 2D, the drug-containing composition consisted of 40 wt/o of the drug 4-amino-5-(4-fluorophenyl)-6,7-dimethoxy-2-[4-(morpholinocarbonyl) perhydro-1,4-diazepin-1-yl]quinoline, (Drug 5) having a solubility of 0.4 mg/mL at pH 7.6, 28 wt % XYLITAB 200, 26 Wt % PEO with an average molecular weight of 600,000 daltons, 5 wt % EXPLOTAB, and 1 Wt % magnesium stearate (see Table A). The drug-containing composition ingredients were first combined without the magnesium stearate and blended for 20 minutes in a TURBULA mixer. The ingredients were milled using a hammer mill and passed through a 0.065-inch screen, then blended again for 20 minutes in the TURBULA mixer. Next, magnesium stearate was added and the drug-containing composition was blended again for 4 minutes in the same mixer.

The water-swellable composition consisted of 74.2 wt % EXPLOTAB, 25.0 wt % PROSOLV 90, 0.3 Wt % Red Lake #40, and 0.5 wt % magnesium stearate. The water-swellable composition ingredients were first combined without the magnesium stearate in a twinshell mixer and blended for 20 minutes. An intensifier bar was inserted into the twinshell mixer and the material was granulated using deionized water. The granules were tray-dried in a 40 □C oven overnight, then milled the following morning using a hammer mill and passed through a 0.065-inch screen. The water-swellable composition ingredients were again placed in a twinshell mixer, the magnesium stearate was added, and the mixture was blended for 4 minutes.

Tablets for Example 2D were compressed and coated as described in Example 1. The resulting tri-layer tablet cores had a total weight of 534 mg and contained a total of 32.58 wt % Drug 6 (174 mg), 22.49 wt % XYLITAB 200, 21.49 wt % PEO 600,000, 17.69 wt % EXPLOTAB, 4.70 wt % PROSOLV 90, 0.06 wt % Red Lake #40, and 0.99 wt % magnesium stearate. The final dry coating weight amounted to 61 mg or 11.4 wt %/o of the tablet core. Five 900 μm diameter holes were then laser-drilled in the coating on each side of the tablet to provide 10 delivery ports per tablet.

Dissolution tests were performed on these tablets in accordance with the procedure described for Example 2A above, with the following exceptions: dissoette stir speed was 50 rpm, and residual drug was analyzed by HPLC using a Phenomenex Luna C₁₈ column with a mobile phase of 60% water/40% acetonitrile/0.1% diethylamine (vol. %) and UV detention at 255 nm. Results are shown in Table 2 and summarized in Table F.

TABLE 2

| | | Drug |
|---|---|---|
| Example | Time (hours) | (% released) |
| 2A | 0 | 0 |
| | 2 | 23 |
| | 4 | 46 |
| | 8 | 85 |
| | 14 | 92 |
| | 20 | 90 |
| 2B | 0 | 0 |
| | 2 | 27 |
| | 4 | 48 |
| | 8 | 72 |
| | 12 | 81 |
| | 18 | 86 |
| | 24 | 83 |
| 2C | 0 | 0 |
| | 2 | 33 |
| | 4 | 50 |
| | 8 | 69 |
| | 14 | 83 |
| | 20 | 85 |
| 2D | 0 | 0 |
| | 2 | 17 |
| | 4 | 41 |
| | 8 | 67 |
| | 14 | 86 |
| | 20 | 90 |

Examples 2A through 2D show greater than 80% drug delivered after 20 hours with virtually no lag time. Along with Example 1, these examples show that different low-solubility drugs can be successfully delivered from dosage forms of this invention.

### EXAMPLE 3

This example demonstrates that the ionic swelling agent can be blended with a high perc ntage of tableting aid to form a tri-layer dosage form with the desired released profile.

For the tablets of Example 3, the drug-containing composition consisted of 35 wt % Drug 1, 30 wt % XYLITAB 200, 29 wt % PEO with an averag molecular weight of 600,000 daltons, 5 wt % EXPLOTAB, and 1 wt % magnesium stearate. The drug-containing composition ingredients were first combined without the magnesium stearate and blended for 20 minutes in a TURBULA mixer. The ingredients were milled using a hammer mill and passed through a 0.065-inch screen, then blended again for 20 minutes in the TURBULA mixer. Next, magnesium stearate was added and the drug-containing composition was blended again for 4 minutes in the same mixer. The drug-containing composition was then wet-granulated using deionized water and dried overnight in a 40 □C oven.

The water-swellable composition consisted of 25 wt % EXPLOTAB, 74.5 wt % PROSOLV 90, and 0.5 wt % magnesium stearate. The water-swellable composition ingredients were first combined without the magnesium stearate and blended for 20 minutes in a TURBULA mixer. Next, magnesium stearate was added and the water-swellable composition was blended again for 4 minutes in the same mixer.

Tablets were compressed and coated as described in Example 1. The final dry coating weight was 48.5 mg (9.7 wt %). Five 900 μm diameter holes were then laser-drilled in the coating on each side of the tablet to provide 10 delivery ports per tablet. Table C summarizes the characteristics of the dosage form.

Dissolution tests were performed on these tablets in accordance with the procedure described for Example 2A, except residual drug was analyzed using the HPLC method described in Example 1. The results are presented in Table 3 and summarized in Table F.

TABLE 3

| | | Drug |
|---|---|---|
| Example | Time (hours) | (wt % released) |
| 3 | 0 | 0 |
| EXPLOTAB/ | 2 | 27 |
| PROSOLV 90 = 25/75* | 4 | 43 |
| | 8 | 65 |
| | 12 | 77 |
| | 19 | 82 |
| | 24 | 93 |

*approximate

The data show that the weight ratio of swelling agent to tableting aid of about 75/25 can be used to achieve a desired drug release profile.

### EXAMPLE 4

This example demonstrates delivery of Drug 1 with the desired release profile from a tri-layer dosage form containing sodium croscarmellose as the ionic swelling agent in the water-swellable composition.

For the tablets of Example 4, the drug-containing composition consisted of 35 wt % Drug 1, 30 wt % XYLITAB 200, 29 wt % PEO with an average molecular weight of 600,000 daltons, 5 wt % EXPLOTAB, and 1 wt % magnesium stearate. The drug-containing composition ingredients were first combined without the magnesium stearate and blended for 20 minutes in a TURBULA mixer. The ingredients were milled using a hammer mill and passed through a 0.065 inch screen, then blended again for 20 minutes in the TURBULA mixer. Next, magnesium stearate was added and the drug-containing composition was blended again for 4 minutes in the same mixer.

For tablets of Example 4, the water-swellable composition consisted of 74.5 wt % sodium croscarmellose (AC-DI-SOL), 25 wt % PROSOLV 90, and 0.5 wt % magnesium stearate. The water-swellable composition ingredients were first combined without the magnesium stearate and blended for 20 minutes in a TURBULA mixer. Next, magnesium stearate was added and the water-swellable composition was blended again for 4 minutes in the same mixer.

Tablets for Example 4 were compressed and coated as described in Example 1. The final dry coating weight was 52 mg (10.4 wt %). Five 900 μm diameter holes were then laser-drilled in the coating on each side of the tablet to provide 10 delivery ports per tablet.

Dissolution tests were performed as described in Example 3 (using the gastric-to-intestinal transfer test of Example 2A with the HPLC method of Example 1). The results are presented in Table 4 and summarized in Table F.

TABLE 4

| | Drug |
|---|---|
| Time (hours) | (wt % released) |
| 0 | 0 |
| 2 | 21 |
| 4 | 48 |
| 8 | 81 |
| 14 | 90 |
| 20 | 89 |

The data show that 21 wt % of the drug was released within 2 hours, 81 wt % within 8 hours, and 89 wt % of the drug was released within 20 hours. Thus, the present invention provided delivery of low-solubility Drug 1 using sodium croscarmellose as the ionic swelling agent.

### EXAMPLE 5

This example demonstrates that high drug loadings may be delivered from tri-layer dosage forms of the invention.

For the tablets of Example 5, the drug-containing composition consisted of 56 wt % Drug 1, 20 wt % XYLITAB 200, 19 wt % PEO with an average molecular weight of 600,000 daltons, 4 wt % EXPLOTAB, and 1 wt % magnesium stearate. The drug-containing composition ingredients were processed as described in Example 4.

The water-swellable composition consisted of 74.5 wt % EXPLOTAB, 25 wt % PROSOLV 90, and 0.5 wt % magnesium stearate. The water-swellable composition ingredients were processed as described in Example 4.

Tablet cores were formed by placing 250 mg of drug-containing composition in a standard 13/32 inch die and gently leveling with the press. Then, 200 mg water-swellable composition was placed in the die on top of the drug-containing composition and leveled. The second half of the drug-containing composition (250 mg) was added and the tablet core compressed to a hardness of about 11 Kp. The resulting tri-layer tablet core had a total weight of 700 mg and contained a total of 40.0 wt % Drug 1 (280 mg), 14.3 wt % XYLITAB 200, 13.6 wt % PEO 600,000 daltons, 24.0 wt % EXPLOTAB, 7.1 wt % PROSOLV 90, and 1.0 wt % magnesium stearate.

Tablets for Example 5 were coated as described in Example 1. The final dry coating weight was 77 mg (11.0 wt %). Five 900 μm diameter holes were then laser-drilled in the coating on each side of the tablet to provide 10 delivery ports per tablet.

Dissolution tests were performed as described in Example 3. The results are presented in Table 5 and summarized in Table F.

TABLE 5

| Time | Drug |
|---|---|
| (hours) | (wt % released) |
| 0 | 0 |
| 2 | 13 |
| 4 | 34 |
| 8 | 63 |
| 14 | 85 |
| 20 | 85 |

The data show that 13 wt % of the drug was released within 2 hours, 63 wt % within 8 hours, and 85 wt % of the drug was released within 20 hours. Thus, the present invention provided delivery of a high dose of low-solubility Drug 1.

### EXAMPLES 6A-6D

These examples demonstrate the relationship between the drug release profile and the water permeability of the coating. For the ti-layer tablets of Examples 6A, 6B, 6C, and 6D, the drug-containing composition consisted of 35 wt % Drug 1, 30 wt % XYLITAB 200, 29 wt % PEO with an average molecular weight of 600,000 daltons, 5 wt % EXPLOTAB, and 1 wt % magnesium stearate. The drug-containing composition ingredients were processed as described in Example 4.

The water-swellable compositions consisted of 74.5 wt % EXPLOTAB, 25 wt % AVICEL PH102, and 0.5 wt % magnesium stearate. The water-swellable composition ingredients were processed as described in Example 4.

Tablets for Examples 6A-6D were compressed and coated as described in Example 1. For the tablets of Example 6A, the coating had a final dry weight of 26 mg (5.2 wt %). For the tablets of Example 6B, the coating had a final dry weight of 49.5 mg (9.9 wt %). For the tablets of Example 6C, the coating had a final dry weight of 78 mg (15.6 wt %). For the tablets of Example 6D, the coating had a final dry weight of 107 mg (21.4 wt %). Five 900 μm diameter holes were then laser-drilled in the coating on each side of the tablet to provide 10 delivery ports per tablet. Table C summarizes the characteristics of the dosage forms.

Generally, the thicker the coating, the lower the expected water permeability. Dissolution tests were performed on these tablets as described in Example 3. Results are shown in Table 6 and are summarized in Table F.

TABLE 6

| | | Drug |
|---|---|---|
| Example | Time (hours) | (wt % released) |
| 6A | 0 | 0 |
| | 2 | 32 |
| | 4 | 58 |
| | 8 | 90 |
| | 14 | 95 |
| | 20 | 94 |
| 6B | 0 | 0 |
| | 2 | 25 |
| | 4 | 40 |
| | 8 | 73 |
| | 14 | 92 |
| | 20 | 92 |
| 6C | 0 | 0 |
| | 2 | 11 |
| | 4 | 36 |
| | 8 | 66 |
| | 14 | 85 |
| | 20 | 92 |
| 6D | 0 | 0 |
| | 2 | 4 |
| | 4 | 27 |
| | 8 | 54 |
| | 14 | 86 |
| | 20 | 90 |

Examples 6A-6D shoe that as the water permeability decreased, i.e., as the coating weight increased, the rate of drug release decreased. The data show that as the coating thickness increased, the fraction of drug delivered between 0 and 8 hours decreased, while the fraction of drug delivered from 8 to 20 hours increased.

### EXAMPLE 7

Exemplary dosage forms of the present invention were made with a tri-layer core geometry of the type depicted in FIG. 1. This example illustrates dosage forms of this invention which release drug over a short duration, utilizing a durable, high permeability coating.

For the tablets of Example 7, the drug-containing composition consisted of 35 wt % Drug 1, 30 wt % XYLITAB 200, 29 wt % PEO with an average molecular weight of 600,000 daltons, 5 wt % EXPLOTAB, and 1 wt % magnesium stearate. The drug-containing composition ingredients were processed as described in Example 4.

The water-swellable composition consisted of 74.5 wt % EXPLOTAB, 25 wt % PROSOLV 90, and 0.5 wt % magnesium stearate. The water-swellable composition ingr dients were processed as described in Example 4.

Tablets were compressed and coated as described in Example 1, except that the coating solution contained CA, PEG 3350, water, and acetone in a weight ratio of 7/3/23/67 (wt %). The amount of water in the coating solution was increased to increase the porosity. The coating had a final dry weight of 56.5 mg (11.3 wt %). Five 900 μm diameter holes were then laser-drilled in the coating on each side of the tablet to provide 10 delivery ports per tablet.

Dissolution tests were performed as described in Example 3, except that the flasks were stirred at 50 rpm. The results are presented in Table 7 and summarized in Table F.

TABLE 7

| Time | Drug |
|---|---|
| (hours) | (wt % released) |
| 0 | 0 |
| 2 | 31 |
| 4 | 66 |
| 8 | 90 |
| 14 | 94 |
| 20 | 94 |

The data show that 31 wt % of Drug 1 was released within 2 hours, 90 wt % within 8 hours, and 94 wt % of the drug was released within 20 hours. Thus, for coatings with increased water permeability, the rate of drug release increased.

### EXAMPLE 8

This example illustrates the delivery of 5-(2-(4-(3-benzisothiazolyl)piperazinyl)ethyl-6-chlorooxindole (Drug 6) having a solubility of 3 □g/mL in model fasted duodenal solution, from a tri-layer dosage form of the invention. The drug was in the form of a solid amorphous dispersion comprising 10 wt % of Drug 6 and 90 wt % hydroxy propylmethyl cellulose acetate succinate, HF grade (HPMCAS-HF), a concentration-enhancing polymer.

Amorphous solid dispersions of Drug 6 in HPMCAS were prepared by spray-drying a solution containing 0.30 wt % Drug 6, 2.7 wt % HPMCAS-HF, and 97 wt % methanol. The solution was spray-dried using a two-fluid external mix spray nozzle at 1.8 bar at a feed rate of 140 g/min into the stainless steel chamber of a Niro spray-dryer, maintained at a temperature of 264 □C at the inlet and 62 □C at the outlet.

To form the drug-containing composition, the following materials were blended: 35 wt % Drug 6 dispersion (1:9 Drug 1:HPMCAS), 29 wt % PEO having an average molecular weight of 600,000 daltons, 30 wt % XYLITAB 200, 5 wt % EXPLOTAB, and 1 wt % magnesium stearate. The drug-containing composition ingredients were first combined without the magnesium stearate and blended for 20 minutes in a TURBULA mixer. Next, half of the magnesium stearate was added and the drug-containing composition was blended again for 4 minutes. The second half of the magnesium stearate was added and the mixture was blended for 5 minutes.

To form the water-swellable composition, the following materials were blended: 74.8 wt % EXPLOTAB, 24.8 wt % PROSOLV 90, and 0.4 wt % magnesium stearate. The water-swellable composition ingredients were processed as described in Example 4.

Tablets for Example 8 were compressed and coated as described in Example 1. Assays of these tablets confirmed 15 mg of active Drug 6 (mgA). The coating had a final dry weight of 43 mg (8.6 wt %). Five 900 μm diameter holes were then laser-drilled in the coating on each side of the tablet to provide 10 delivery ports per tablet. Table C summarizes the characteristics of the dosage form.

Release of the Drug 6 dispersion from the tri-layer tablets into simulated intestinal buffer was measured. The dissoette flasks were stirred at 50 rpm at 37 □C. For each sampling interval, a tablet was removed from the test solution, placed in 200 mL of recovery solution consisting of 75% methanol/25% water, and stirred overnight to dissolve the remaining drug in the tablet. Residual drug was analyzed by HPLC using a Phenomenex ODS 20 column. The mobile phase consisted of 60% 0.02 M KH₂PO₄, pH 3/40% acetonitrile. Drug concentration was calculated by comparing UV absorbance at 254 nm to the absorbance of Drug 6 standards. The amount of drug remaining in the tablets was subtracted from the total initial amount of drug in the tablet to obtain the amount released at each time interval. The results are presented in Table 8 and summarized in Table F.

TABLE 8

| Time | Drug |
|---|---|
| (hours) | (wt % released) |
| 0 | 0 |
| 1 | 10 |
| 2 | 23 |
| 4 | 48 |
| 8 | 77 |
| 12 | 88 |
| 18 | 85 |
| 24 | 89 |

The data demonstrate satisfactory delivery of a dispersion of Drug 6 from tri-layer dosage forms of this invention.

### EXAMPLE 9

### This example describes the results of tests to determine the swelling volume of swelling agents that may be used in the formulation of the water-swellable composition.

The following experiment was used to determine the swelling ratio of materials. The materials were first blended and then 500 mg of the material was compressed into a tablet using a 13/32-inch die, the tablet having a strength ranging from 3 to 16 Kp/cm². This compressed material was then placed into a glass cylinder of approximately the same inside diameter as the tablet. The height of the tablet was then measured. Using this height and the diameter of the tablet, the volume of the dry material was determined. Next, the glass cylinder was filled with test media of either deionized water, simulated intestinal buffer, or simulated gastric buffer. The glass cylinder and test media were all equilibrated at a constant temperature of 37 □C. As the materials in the tablet absorbed water, the height of the tablet increased. At each time interval, the height of the tablet was measured, from which the volume of the swollen tablet was determined. The ratio of the volume of the tablet after reaching a constant height to that of the volume of the dry tablet is the swelling ratio of the material. The results of these tests are shown in Table 9.

TABLE 9

| Water-Swellable Composition | | | | | |
|---|---|---|---|---|---|
| | | Swelling | Swelling Ratio (v/v) | | |
| | Tableting | Agent/ | | Intes- | |
| Swelling | Aid/ | Tableting | Gastric | tinal | Wa- |
| Agent | Additive | Aid (w/w) | Buffer | Buffer | ter |
| PEO 5,000,000 | NONE | 100/0 | 2.4 | 2.4 | 2.4 |
| PEO 5,000,000 | Microcrystal-line | 85/15 | 2.2 | 2.1 | 2.4 |
| | cellulose¹ | | | | |
| PEO 5,000,000 | Microcrystal-line | 70/30 | 2.0 | 2.1 | 2.4 |
| | cellulose | | | | |
| PEO 5,000,000 | Microcrystal-line | 50/50 | 2.0 | 1.9 | 1.9 |
| | cellulose | | | | |
| PEO 5,000,000 | NaCl | 70/30 | 2.6 | 2.6 | 2.8 |
| PEO 2,000,000 | Microcrystal-line | 85/15 | 2.8 | 2.8 | 3.0 |
| | cellulose | | | | |
| Polyacrylic | Silicified | 70/30 | 1.9 | 1.5 | — |
| acid² | microcrystal-line | | | | |
| | cellulose³ | | | | |
| Polyacrylic | Microcrystal-line | 50/50 | 1.8 | 1.7 | — |
| acid | cellulose | | | | |
| Sodium cros- | None | 100/0 | 7.0 | 5.4 | 7.1 |
| carmelose⁴ | | | | | |
| Sodium cros- | Microcrystal-line | 85/15 | 7.1 | 5.9 | 7.2 |
| carmellose | cellulose | | | | |
| Sodium cros- | Microcrystal-line | 70/30 | 5.5 | 6.3 | 5.5 |
| carmellose | cellulose | | | | |
| Sodium cros- | Microcrystal-line | 50/50 | 4.6 | 5.3 | 5.7 |
| carmellose | cellulose | | | | |
| Sodium starch | Microcrystal-line | 50/50 | 7.1 | 7.7 | 25.2 |
| glycolate⁵ | cellulose | | | | |
| Sodium starch | Microcrystal-line | 70/30 | 9.0 | 9.6 | 26.8 |
| glycolate | cellulose | | | | |
| Sodium starch | Microcrystal-line | 85/15 | 10.9 | 11.9 | 34.7 |
| glycolate | cellulose | | | | |
| Sodium starch | Silicified | 50/50 | 7.9 | 8.7 | — |
| glycolate | Microcrystal-line | | | | |
| | cellulose | | | | |
| Sodium starch | Silicified | 75/25 | 7.4 | 9.1 | 14.4 |
| glycolate | Microcrystal-line | | | | |
| | cellulose | | | | |
| Sodium starch | Silicified | 70/30 | 10.6 | 11.2 | — |
| glycolate | Microcrystal-line | | | | |
| | cellulose | | | | |
| Sodium starch | Hydroxypropyl | 98/2 | — | 17.2 | — |
| glycolate | cellulose⁶ | | | | |
| Sodium starch | Hydroxypropyl | 95/5 | 5.6 | 8.4 | — |
| glycolate | cellulose | | | | |
| Sodium starch | Hydroxypropyl | 90/10 | 7.2 | 6.9 | — |
| glycolate | cellulose | | | | |
| Sodium starch | Hydroxypropyl | 85/15 | — | 3.8 | 3.8 |
| glycolate | cellulose | | | | |
| Sodium starch | Hydroxypropyl | 70/30 | 3.7 | 3.9 | 3.3 |
| glycolate | cellulose | | | | |
| Sodium starch | Hydroxypropyl | 50/50 | 2.4 | 2.5 | 2.4 |
| glycolate | cellulose | | | | |
| Sodium | Silicified | 50/50 | 2.7 | 2.9 | — |
| alginate⁷ | microcrystal-line | | | | |
| | cellulose | | | | |
| Hydroxyethyl | NONE | 100/0 | 2.8 | 2.8 | 2.7 |
| cellulose⁸ | | | | | |
| Hydroxyethyl | Microcrystal-line | 50/50 | 2.4 | 2.1 | 2.5 |
| cellulose | cellulose | | | | |

¹= AVICEL
²= CARBOPOL 974PNF
³= PROSOLV 90
⁴= AC-DI-SOL
⁵= EXPLOTAB
⁶= Klucel
⁷= Keltone LVCR
⁸= Natrosol

### EXAMPLES 10A-10C

These examples demonstrate that various osmogens can be used in the drug-containing composition to form tri-layer dosage forms with the desired release profile. For the tablets of Example 10A, the drug-containing composition consisted of 35 wt % Drug 1, 29 wt % PEO having an average molecular weight of 600,000 daltons, 30 wt % sorbitol, 5 wt % EXPLOTAB, and 1 wt % magnesium stearate. For the tablets of Example 10B, the drug-containing composition consisted of 35 wt % Drug 1, 29 wt % PEO having an average molecular weight of 600,000 daltons, 30 wt % FAST FLO Lactose, 5 wt % EXPLOTAB, and 1 wt % magnesium stearate. For the tablets of Example 10C, the drug-containing composition consisted of 35 wt % Drug 1, 19 wt % PEO having an average molecular weight of 600,000 daltons, 40 wt % XYLITAB 200, 5 wt % EXPLOTAB, and 1 wt % magnesium stearate. The drug-containing composition ingredients were processed as described in Example 4.

For the tablets of Examples 10A-10C, the water-swellable compositions consisted of 74.5 wt % EXPLOTAB, 25.0 wt % PROSOLV 90, and 0.5 wt % magnesium stearate. For the tablets of Example 10C, the water-swellable composition ingredients were processed as described in Example 4. For the tablets of Examples 10A and 10B, the water-swellable composition ingredients were processed as described in Example 1.

Tablets for Examples 10A-10B were compressed and coated as described in Example 1. The final dry coating weights for each example were 58 mg (11.6 wt %) for 10A, 35 mg (7.0 wt %) for 10B, and 48.5 mg (9.7 wt %) for 10C respectively. For all of these examples, five 900 μm diameter holes were then laser-drilled in the coating on each side of the tablet to provide 10 delivery p rts per tablet. Table C summarizes the characteristics of the dosage forms.

Dissolution tests were performed as described in Example 3, except that the flasks for Examples 10A-10C were stirred at 50 rpm. The results are presented in Table 13 and summarized in Table F.

TABLE 10

| | | Drug |
|---|---|---|
| Example | Time (hours) | (wt % released) |
| 10A | 0 | 0 |
| 30% Sorbitol | 1 | 4 |
| | 2 | 20 |
| | 4 | 40 |
| | 6 | 53 |
| | 8 | 68 |
| | 14 | 86 |
| | 20 | 90 |
| 10B | 0 | 0 |
| 30% Lactose | 2 | 11 |
| | 4 | 35 |
| | 8 | 60 |
| | 12 | 90 |
| | 18 | 89 |
| | 20 | 90 |
| | 24 | 90 |
| 10C | 0 | 0 |
| 40% XYLITAB | 1 | 12 |
| | 2 | 30 |
| | 4 | 48 |
| | 6 | 77 |
| | 8 | 81 |
| | 14 | 89 |
| | 20 | 89 |

The data show that a variety of materials may be used as the osmogen in the drug-containing composition without any adverse effect on the desired drug release profile.

### EXAMPLE 11

This example illustrates delivery of two different drugs from a tri-layer dosage form of the invention. Tri-layer tablets for Example 11 were made with two different drug layers.

For the tablets of Example 11, the top drug-containing composition consisted of 17 wt % cetirizine dihydrochloride (Drug 7), 25 wt % PROSOLV 90, 40 wt % XYLITAB 200, 17 wt % EXPLOTAB, and 1 wt % magnesium stearate. The top layer did not contain a drug entraining agent (e.g., PEO), which reduced the viscosity of th solvated layer and allow d faster release of Drug 7. The bottom drug-containing composition consisted of 60 wt % pseudoephedrine hydrochloride (Drug 8), 34 wt % PEO having an average molecular weight of 600,000, 5 wt % EXPLOTAB, and 1 wt % magnesium stearate. Each mixture of drug-containing composition ingredients was processed as described in Example 4. The water-swellable composition consisted of 74.5 wt % EXPLOTAB, 25 wt % PROSOLV 90, and 0.5 wt % magnesium stearate. The water-swellable composition ingredients were processed as described in Example 1.

Tablets for Example 11 were compressed as described in Example 1, except that 400 mg of the bottom layer containing pseudoephedrine was placed in the f-press and leveled, 100 mg of the sweller layer was added and leveled, and 60 mg of the top layer containing cetirizine was added and the tablet compressed. Tablets were coated as described in Example 1. The final dry coating weight for Example 11 was 125.5 mg (22.4 wt %). Five 900 μm diameter holes were then laser-drilled in the coating on the pseudoephedrine side of the tablet, and five 2000 μm diameter holes were laser-drilled in the coating on the cetirizine side of the tablet, to provide 10 delivery ports per tablet.

Dissolution tests were performed as described in Example 3, except that the flasks for Example 11 were stirred at 50 rpm, and the recovery solution for dissolution of residual drug was 50% acetonitrile/50% water for Example 11. The HPLC method for analysis of pseudoephedrine and cetirizine uses a Zorbax Stablebond® CN column with a mobile phase of 50% 0.1 M KH₂PO₄, pH 6.5/50% methanol containing 1 g/L sodium octanesulfonate, and UV detection at 214 nm. The results are presented in Table 11 and summarized in Table F.

TABLE 11

| | | Drug |
|---|---|---|
| Example | Time (hours) | (wt % released) |
| 11 | 0 | 0 |
| Drug 7 | 0.5 | 23 |
| | 1 | 47 |
| | 2 | 52 |
| | 4 | 56 |
| | 8 | 97 |
| | 12 | 97 |
| | 18 | 97 |
| | 24 | 97 |
| 11 | 0 | 0 |
| Drug 8 | 0.5 | 0 |
| | 1 | 5 |
| | 2 | 17 |
| | 4 | 32 |
| | 8 | 64 |
| | 12 | 74 |
| | 18 | 97 |
| | 24 | 98 |

The data show that two different drugs can be successfully delivered from tri-layer dosage forms of the invention, and that the rate of delivery for each drug can be independently modified.

### EXAMPLES 12A-12C

Examples 12A-12C illustrate the delivery of a low solubility drug (Drug 1) using three different dosage form geometries, each comprising a drug-containing composition and a water-swellable composition.

Tablets for Example 12A were tri-layer dosage forms, with the drug-containing composition consisting of 35 wt % Drug 1, 30 wt % XYLITAB 200, 29 wt % PEO with an average molecular weight of 600,000 daltons, 5 wt % EXPLOTAB, and 1 wt % magnesium stearate. The drug-containing composition ingredients were processed as described in Example 4. The water-swellable composition consisted of 74.5 wt % EXPLOTAB, 25 wt % AVICEL PH200, and 0.5 wt % magnesium stearate. The water-swellable composition ingredients were processed as described in Example 4. Tablets were compressed and coated as described in Example 1. The coating had a final dry weight of 52.5 mg (10.5 wt %). Five 900 μm diameter holes were then laser-drilled in the coating on each side of the tablet to provide 10 delivery ports per tablet.

Tablets for Example 12B were concentric core dosage forms, with the same drug-containing composition and water-swellable composition as Example 12A, blended using the same processes. To form the tablets, 100 mg of the water-swellable composition was compressed with ¼-inch tooling to a hardness of 6 Kp. Next, 200 mg of the drug-containing composition was placed in the f-press and gently leveled and compressed with a spatula. The sweller core was placed on top of this and centered. The remaining drug-containing composition (200 mg) was added and the tablet compressed with 9/16-inch tooling to a hardness of about 11 Kp. Tablets were coated as described in Example 1. The coating had a final dry weight of 55 mg (11.0 wt %). Five 900 μm diameter holes were then laser-drilled in the coating on each side of the tablet to provide 10 delivery ports per tablet.

Tablets for Example 12C were homogeneous core dosage forms (as in FIG. 4). The tablet cores contained 28 wt % Drug 1, 21 wt % XYLITAB 200, 20 wt % PEO with an average molecular weight of 600,000 daltons, 30 wt % EXPLOTAB, and 1 wt % magnesium stearate. The homogeneous core ingredients were first combined without the magnesium stearate and blended for 20 minutes in a TURBULA mixer. The ingredients were milled using a hammer mill and passed through a 0.065-inch screen, then blended again for 20 minutes in the TURBULA mixer. Next, magnesium stearate was added and the composition was blended again for 4 minutes in the same mixer. Tablets contained 500 mg each. Tablets were coated as described in Example 1. The coating had a final dry weight of 47.5 mg (9.5 wt %). Five 900 μm diameter holes were then laser-drilled in the coating on each side of the tablet to provide 10 delivery ports per tablet.

Dissolution tests for Examples 12A-12C were performed as described in Example 3. The results are presented in Table 12 and summarized in Table F.

TABLE 12

| | | Drug |
|---|---|---|
| Example | Time (hours) | (wt % released) |
| 12A | 0 | 0 |
| | 2 | 25 |
| | 4 | 53 |
| | 8 | 75 |
| | 14 | 95 |
| | 20 | 95 |
| 12B | 0 | 0 |
| | 2 | 27 |
| | 4 | 49 |
| | 8 | 69 |
| | 14 | 87 |
| | 20 | 88 |
| 12C | 0 | 0 |
| | 2 | 11 |
| | 4 | 40 |
| | 8 | 65 |
| | 14 | 81 |
| | 20 | 85 |

The data show that drug can be delivered from dosage forms of the invention in various geometries, with no time lag and low residual drug.

### EXAMPLE 13

This example demonstrates delivery of Drug 1 with the desired releases profile from a concentric core dosage form containing sodium croscarmellose as the ionic swelling agent.

For the tablets of Example 13, the drug-containing composition consisted of 35 wt % XYLITAB 200, 29 wt % PEO with an average molecular weight of 600,000 daltons, 5 wt % EXPLOTAB, and 1 wt % magnesium stearate. The drug-containing composition ingredients were processed as described in Example 4.

For tablets of Example 13, the water-swellable composition consisted of 74.5 wt % sodium croscarmellose, 25 wt % PROSOLV 90, and 0.5 wt % magnesium stearate. The water-swellable composition ingredients were processed as described in Example 4.

To form the tablets, 100 mg of the water-swellable composition was compressed with ¼-inch tooling to a hardness of 5 Kp. Next, 200 mg of the drug-containing composition was placed in the f-press and gently leveled and compressed with a spatula. The sweller core was placed on top of-this and centered. The remaining drug-containing composition (200 mg) was added and the tablet compressed with 9/16-inch tooling to a hardness of about 11 Kp. Tablets were coated as described in Example 1. The coating had a final dry weight of 50 mg (10.0 wt %). Five 900 μm diameter holes were then laser-drilled in the coating on each side of the tablet to provide 10 delivery ports per tablet.

Dissolution tests were performed as described in Example 3. The results are presented in Table 13 and summarized in Table F.

TABLE 13

| | Drug |
|---|---|
| Time (hours) | (wt % released) |
| 0 | 0 |
| 2 | 21 |
| 4 | 54 |
| 8 | 75 |
| 14 | 85 |
| 20 | 84 |

The data show that 21 wt % of the drug was released within 2 hours, 75 wt % within 8 hours, and 84 wt % of the drug was released within 20 hours.

### EXAMPLE 14

### This example demonstrates delivery of Drug 1 with the desired release profile from a granular core dosage form containing a granular swelling agent.

The tablets contained 28 wt % Drug 1, 24 wt % XYLITAB 200, 23 wt % PEO with an average molecular weight of 600,000 daltons, 24 wt % EXPLOTAB (granular, 0.85-1.18 mm), and 1 wt % magnesium stearate. The mixture was processed using the same procedures used to process the drug-containing composition of Example 4. Tablets contained 500 mg each. Tablets were coated as described in Example 1. The coating had a final dry weight of 47.5 mg (9.5 wt %). Five 900 μm diameter holes were then laser-drilled in the coating on each side of the tablet to provide 10 delivery ports per tablet.

Dissolution tests were performed as described in Example 3. The results are presented in Table 14 and summarized in Table F.

TABLE 14

| | Drug |
|---|---|
| Time (hours) | (wt % released) |
| 0 | 0 |
| 2 | 20 |
| 4 | 45 |
| 8 | 69 |
| 14 | 81 |
| 20 | 85 |

The data show that 20 wt % of the drug was released within 2 hours, 69 wt % within 8 hours, and 85 wt % of the drug was released within 20 hours. Thus, the present invention provided delivery of a low-solubility drug from a granular core dosage form using granular EXPLOTAB as the swelling agent.

### EXAMPLE 15

This example demonstrates the in vivo release of Drug 2 from a granular core dosage form. The tablets of Example 15 contained 22.5 wt % Drug 2, 30 wt % XYLITAB 200, 26.5 wt % PEO with an average molecular weight of 600,000 daltons, 20 wt % EXPLOTAB (granular, 0.85-1.18 mm), and 1 wt % magnesium stearate. The mixture was processed using the same procedures used to process the drug-containing composition of Example 4. Tablets contained 500 mg each. Tablets were coated as described in Example 1. The coating had a final dry weight of 55.5 mg (11.1 wt %). Eight 1000 μm diameter slits were then laser-drilled in the coating on the band of the tablet to provide delivery ports.

In viva residual tests were performed in 5 dogs as follows: Each of five dogs were dosed with tablets (which were marked for later identification) over a six-hour period (i.e., one tablet every two hours) with oral gavage of 50 mL water. The bowel movement was screened for tablets and the recovery time noted. All tablets were recovered intact, i.e., there were no splits in the coatings. The amount of undelivered drug was determined by extracting the unreleased drug from the tablets and the drug released was determined by subtracting the unreleased amount from the known initial amount of drug present in the tablets. Results are shown in Table 15.

TABLE 15.1

| Dog | | Drug |
|---|---|---|
| No. | Time (hours) | (wt % released) |
| 1 | 7.75 | 51 |
| | 5.75 | 27 |
| | 3.75 | 15 |
| 2 | 24 | 75 |
| | 22 | 66 |
| | 20 | 71 |
| 3 | 7.5 | 47 |
| | 5.5 | 30 |
| | 3.5 | 28 |
| 4 | 7.5 | 48 |
| | 5.5 | 33 |
| | 3.5 | 25 |
| 5 | 28 | 68 |
| | 26 | 74 |
| | 24 | 68 |

These tablets were also tested in vitro using a residual dissolution test. These tests were performed in a USP type 2 dissoette using the conditions described in Example 2A. Results are shown in Table 15.2.

TABLE 15.2

| | Drug |
|---|---|
| Time (hours) | (wt % released) |
| 0 | 0 |
| 2 | 22 |
| 4.5 | 52 |
| 8.3 | 61 |
| 14 | 65 |
| 20 | 71 |

The data show satisfactory in vivo drug delivery with dosage forms of the invention. Good correlation is observed between in vitro and in vivo data.

### EXAMPLE 16

This example demonstrates the in vivo delivery of Drug 2 from tri-layer tablets. For the tablets of Example 16, the drug-containing composition consisted of 28 wt % Drug 2, 37 wt % XYLITAB 200, 29 wt % PEO with an average molecular weight of 600,000 daltons, 5 wt % EXPLOTAB, and 1 wt % magnesium stearate; and the water-swellable composition consisted of 72.5 wt % EXPLOTAB, 25 wt % AVICEL PH102, and 2.5 wt % magnesium stearate. The drug-containing compositions and water-swellable composition were processed as described in Example 4. Tablets were compressed and coated as described in Example 1. The coating had a final dry weight of 50.5 mg (10.1 wt %). Five 900 μm diameter holes were then laser-drilled in the coating on each side of the tablet to provide 10 delivery ports per tablet.

In vivo residual tests were performed in dogs as follows: Each of five dogs were dosed with tablets (which were marked for later identification) over a six-hour period (i.e., one tablet every two hours) with oral gavage of 50 mL water. The bowel movement was screened for tablets and the recovery time noted. All tablets were recovered intact, i.e., there were no splits in the coatings. The amount of undelivered drug was determined by extracting the unreleased drug from the tablets and the drug released was determined by subtracting the unreleased amount from the known initial amount of drug present in the tablets. Results are shown in Table 16.1.

TABLE 16.1

| Dog | | Drug |
|---|---|---|
| No. | Time (hours) | (wt % released) |
| 1 | 24 | 86 |
| | 22 | 86 |
| | 20 | 84 |
| 2 | 26.5 | 87 |
| | 24.5 | 87 |
| | 22.5 | 86 |
| 3 | 26.5 | 86 |
| | 24.5 | 86 |
| | 22.5 | 85 |
| 4 | 33-48 | 87 |
| | 31-46 | 90 |
| | 29-44 | 87 |
| 5 | 26.5 | 88 |
| | 24.5 | 85 |
| | 22.5 | 82 |

These tablets were also tested in vitro using a residual dissolution test. These tests were performed in a USP type 2 dissoette using the conditions described in Example 2A. Results are shown in Table 16.2.

TABLE 16.2

| | Drug |
|---|---|
| Time (hours) | (wt % released) |
| 0 | 0 |
| 2 | 23 |
| 4 | 46 |
| 8 | 85 |
| 14 | 92 |
| 20 | 90 |

The data show satisfactory in vivo drug delivery with dosage forms of the invention. Good correlation is observed between in vitro and in vivo data.

The terms and expressions which have been employed in the foregoing specification are used therein as terms of description and not of limitation, and there is no intention, in the use of such terms and expressions, of excluding equivalents of the features shown and described or portions thereof, it being recognized that the scope of the invention is defined and limited only by the claims which follow.

TABLE A
Composition of the Drug-containing Layer for “Trilayer” and Concentric Core Examples

| | | Drug-containing Layer Composition | | | | | | | | |
|---|---|---|---|---|---|---|---|---|---|---|
| | | | | | | | Mg | | | |
| | | Drug | | PEO | Explotab | Xylitab 200 | Stearate | | | |
| | | Conc. | PEO | Conc. | Conc. | Conc. | Conc. | Other | Conc. | Processing |
| Example | Drug | (wt %) | Type | (wt %) | (wt %) | (wt %) | (wt %) | Ingredients | (wt %) | Method |
| 1 | 1 | 35 | 600K | 29 | 5 | 30 | 1 | — | — | Wet Granulated |
| 2A | 2 | 28 | 600K | 29 | 5 | 37 | 1 | — | — | Dry Blended |
| 2B | 3 | 33 | 600K | 30 | 5 | 31 | 1 | — | — | Dry Blended |
| 2C | 4 | 35 | 600K | 29 | 5 | 30 | 1 | — | — | Dry Blended |
| 2D | 5 | 40 | 600K | 26 | 5 | 28 | 1 | — | — | Dry Blended |
| 3 | 1 | 35 | 600K | 29 | 5 | 30 | 1 | — | — | Wet Granulated |
| 4 | 1 | 35 | 600K | 29 | 5 | 30 | 1 | — | — | Dry Blended |
| 5 | 1 | 56 | 600K | 19 | 4 | 20 | 1 | — | — | Dry Blended |
| 6A | 1 | 35 | 600K | 29 | 5 | 30 | 1 | — | — | Dry Blended |
| 6B | 1 | 35 | 600K | 29 | 5 | 30 | 1 | — | — | Dry Blended |
| 6C | 1 | 35 | 600K | 29 | 5 | 30 | 1 | — | — | Dry Blended |
| 6D | 1 | 35 | 600K | 29 | 5 | 30 | 1 | — | — | Dry Blended |
| 7 | 1 | 35 | 600K | 29 | 5 | 30 | 1 | — | — | Dry Blended |
| 8 | 6 | 3.5 | 600K | 29 | 5 | 30 | 1 | HPMCAS-HF | 31.5 | Dry Blended |
| | | Dispersion | | | | | | | | |
| 10A | 1 | 35 | 600K | 29 | 5 | 30 sorbitol | 1 | — | — | Dry Blended |
| 10B | 1 | 35 | 600K | 29 | 5 | 30 lactose | 1 | — | — | Dry Blended |
| 10C | 1 | 35 | 600K | 19 | 5 | 40 | 1 | — | — | Dry Blended |
| 11(1) | 7 | 17 | — | 0 | 17 | 40 | 1 | PROSOLV | 25 | Dry Blended |
| 11(2) | 8 | 60 | 600K | 34 | 5 | 0 | 1 | — | — | Dry Blended |
| 12A | 1 | 35 | 600K | 29 | 5 | 30 | 1 | — | — | Dry Blended |
| 12B | 1 | 35 | 600K | 29 | 5 | 30 | 1 | — | — | Dry Blended |
| 13 | 1 | 35 | 600K | 29 | 5 | 30 | 1 | — | — | Dry Blended |
| 16 | 2 | 28 | 600K | 29 | | 37 | 1 | — | — | Dry Blended |

TABLE B
Composition of the Water-swellable Composition for Trilayer
and Concentric Core Examples

| | | | | | Mg | | | |
|---|---|---|---|---|---|---|---|---|
| | | Sweller | Tabletting | Tabletting | Stearate | | | |
| | Sweller | Conc. | Aid | Aid Conc. | Conc. | Other | Conc. | |
| Example | Type | (wt %) | Type | (wt %) | (wt %) | Ingredients | (wt %) | Processing Method |
| 1 | Explotab | 74.5 | Prosolv 90 | 24.5 | 1.0 | — | — | Wet Granulated |
| 2A | Explotab | 72.5 | Avicel | 25 | 2.5 | — | — | Dry Blended |
| 2B | Explotab | 74.5 | Prosolv 90 | 24.5 | 1.0 | — | — | Wet Granulated |
| 2C | Explotab | 74.5 | Avicel | 25 | 0.5 | — | — | Dry Blended |
| 2D | Explotab | 74.2 | Prosolv 90 | 25 | 0.5 | Red Lake #40 | 0.3 | Wet Granulated |
| 3 | Explotab | 25 | Prosolv 90 | 74.5 | 0.5 | — | — | Dry Blended |
| 4 | sodium | 74.5 | Prosolv 90 | 25 | 0.5 | — | — | Dry Blended |
| | croscar- | | | | | | | |
| | mellose | | | | | | | |
| 5 | Explotab | 74.5 | Prosolv 90 | 25 | 0.5 | — | — | Dry Blended |
| 6A | Explotab | 74.5 | Prosolv 90 | 25 | 0.5 | — | — | Dry Blended |
| 6B | Explotab | 74.5 | Prosolv 90 | 25 | 0.5 | — | — | Dry Blended |
| 6C | Explotab | 74.5 | Prosolv 90 | 25 | 0.5 | — | — | Dry Blended |
| 6D | Explotab | 74.5 | Prosolv 90 | 25 | 0.5 | — | — | Dry Blended |
| 7 | Explotab | 74.5 | Prosolv 90 | 25 | 0.5 | — | — | Dry Blended |
| 8 | Explotab | 74.8 | Prosolv 90 | 24.8 | 0.4 | — | — | Dry Blended |
| 10A | Explotab | 74.5 | Prosolv 90 | 25 | 0.5 | — | — | Dry Blended |
| 10B | Explotab | 74.5 | Prosolv 90 | 25 | 0.5 | — | — | Dry Blended |
| 10C | Explotab | 74.5 | Prosolv 90 | 25 | 0.5 | — | — | Dry Blended |
| 11 | Explotab | 74.5 | Prosolv 90 | 25 | 0.5 | — | — | Dry Blended |
| 12A | Explotab | 74.5 | Prosolv 90 | 25 | 0.5 | — | — | Dry Blended |
| 12B | Explotab | 74.5 | Avicel | 25 | 0.5 | — | — | Dry Blended |
| 13 | sodium | 74.5 | Prosolv 90 | 25 | 0.5 | — | — | Dry Blended |
| | croscar- | | | | | | | |
| | mellose | | | | | | | |
| 16 | Explotab | 72.5 | Avicel | 25 | 2.5 | — | — | Dry Blended |

TABLE C
Details of Tablet Formulations for Trilayer and Concentric Core Examples

| | | Ratio of Total | | | | Coating Amount | | |
|---|---|---|---|---|---|---|---|---|
| | Core | Drug Layers to | CA | PEG | H₂O | (wt % of | | |
| | Weight | Sweller Layer | Conc. | Cont. | Conc. | uncoated | Number | Hole Size |
| Example | (mg) | (w/w) | (wt %) | (wt %) | (wt %) | tablet) | of Holes | (μm) |
| 1 | 500 | 4:1 | 7 | 3 | 5 | 9.5 | 10 | 900 |
| 2A | 500 | 4:1 | 7 | 3 | 5 | 10.1 | 10 | 900 |
| 2B | 500 | 4:1 | 7 | 3 | 5 | 9.3 | 10 | 900 |
| 2C | 500 | 4:1 | 7 | 3 | 5 | 9.1 | 10 | 900 |
| 2D | 534 | 4:1 | 7 | 3 | 5 | 11.4 | 10 | 900 |
| 3 | 500 | 4:1 | 7 | 3 | 5 | 9.7 | 10 | 900 |
| 4 | 500 | 4:1 | 7 | 3 | 5 | 10.4 | 10 | 900 |
| 5 | 500 | 2.5:1 | 7 | 3 | 5 | 11.0 | 10 | 900 |
| 6A | 500 | 4:1 | 7 | 3 | 5 | 5.2 | 10 | 900 |
| 6B | 500 | 4:1 | 7 | 3 | 5 | 9.9 | 10 | 900 |
| 6C | 500 | 4:1 | 7 | 3 | 5 | 15.6 | 10 | 900 |
| 6D | 500 | 4:1 | 7 | 3 | 5 | 21.4 | 10 | 900 |
| 7 | 500 | 4:1 | 7 | 3 | 23 | 11.3 | 10 | 900 |
| 8 | 500 | 4:1 | 7 | 3 | 5 | 8.6 | 10 | 900 |
| 10A | 500 | 4:1 | 7 | 3 | 5 | 11.6 | 10 | 900 |
| 10B | 500 | 4:1 | 7 | 3 | 5 | 7.0 | 10 | 900 |
| 10C | 500 | 4:1 | 7 | 3 | 5 | 9.7 | 10 | 900 |
| 11 | 500 | 4.6:1 | 7 | 3 | 5 | 22.4 | 10 | 2000, 900 |
| 12A | 500 | 4:1 | 7 | 3 | 5 | 10.5 | 10 | 900 |
| 12B | 500 | 4:1 | 7 | 3 | 5 | 11.0 | 10 | 900 |
| 13 | 500 | 4:1 | 7 | 3 | 5 | 10.0 | 10 | 900 |
| 16 | 500 | 4:1 | 7 | 3 | 5 | 10.1 | 10 | 900 |

TABLE D
Composition of the Core for “Granular Core” and
Homogeneous Core Examples

| | | Drug-containing Layer Composition | | | | | | |
|---|---|---|---|---|---|---|---|---|
| | | | | | | | Mg | |
| | | Drug | | PEO | Explotab | Xylitab 200 | Stearate | |
| | | Conc. | PEO | Conc. | Conc. | Conc. | Conc. | Processing |
| Example | Drug | (wt %) | Type | (wt %) | (wt %) | (wt %) | (wt %) | Method |
| 12C | 1 | 28 | 600K | 29 | 20 | 22 | 1 | Dry Blended |
| 14 | 1 | 28 | 600K | 23 | 24 | 24 | 1 | Dry Blended |
| | | | | | granular | | | |
| 15 | 2 | 22.5 | 600K | 26.5 | 20 | 30 | 1 | Dry Blended |
| | | | | | granular | | | |

TABLE E
Details of Tablet Formulations for “Granular Core”
and Homogeneous Core Examples

| | | | | | | Coating Amount | | |
|---|---|---|---|---|---|---|---|---|
| | Core | | CA | PEG | H2O | (wt % of | | |
| | Weight | Sweller | Conc. | Cont. | Conc. | uncoated | Number | Hole Size |
| Example | (mg) | (wt % of core) | (wt %) | (wt %) | (wt %) | tablet) | of Holes | (□m) |
| 12C | 500 | 20 | 7 | 3 | 5 | 9.5 | 10 | 900 |
| 14 | 500 | 24 | 7 | 3 | 5 | 9.5 | 10 | 900 |
| 15 | 500 | 20 | 7 | 3 | 5 | 11.1 | 8 | 1000 |
| | | | | | | | | slits |

TABLE F
Summary of Release Rates For All Examples

| | | | | | | Release |
|---|---|---|---|---|---|---|
| | 2-hr | 8-hr | 12-hr | 16-hr | 20-hr | Rate |
| | Release | Release | Release | Release | Release | 2-12 hr |
| Example | (%) | (%) | (%) | (%) | (%) | (%/hr) |
| 1 | 19 | 76* | 94 | 95* | 100 (24 hr) | 7.5 |
| 2A | 23 | 85 | 90* | 91* | 90 | 6.7 |
| 2B | 27 | 72 | 81 | 84* | 83 (24 hr) | 5.4 |
| 2C | 33 | 69 | 78* | 84* | 85 | 4.5 |
| 2D | 17 | 67 | 80* | 87* | 90 | 6.3 |
| 3 | 27 | 65 | 77 | 80* | 93 (24 hr) | 5.0 |
| 4 | 21 | 81 | 87* | 90* | 89 | 6.6 |
| 5 | 13 | 63 | 78* | 85* | 85 | 6.5 |
| 6A | 32 | 90 | 93* | 95* | 94 | 6.1 |
| 6B | 25 | 73 | 86* | 92* | 92 | 6.1 |
| 6C | 11 | 66 | 79* | 87* | 92 | 6.8 |
| 6D | 4 | 54 | 75* | 87* | 90 | 7.1 |
| 7 | 31 | 90 | 93* | 94* | 94 | 6.2 |
| 8 | 23 | 77 | 88 | 88* | 89 (24 hr) | 6.5 |
| 10A | 20 | 68 | 80* | 87* | 90 | 6.0 |
| 10B | 11 | 60 | 90 | 89* | 90 (24 hr) | 7.9 |
| 10C | 30 | 81 | 86* | 89* | 89 | 5.6 |
| 11 | 23 | 97 | 97 | 97* | 97 (24 hr) | 7.4 |
| Drug 7 | | | | | | |
| 11 | 17 | 64 | 74 | 89* | 98 (24 hr) | 5.7 |
| Drug 8 | | | | | | |
| 12A | 25 | 75 | 88* | 95* | 95 | 6.3 |
| 12B | 27 | 69 | 81* | 87* | 88 | 5.4 |
| 12C | 11 | 65 | 76* | 82* | 85 | 6.5 |
| 13 | 21 | 75 | 88* | 85* | 84 | 6.7 |
| 14 | 20 | 69 | 77* | 82* | 85 | 5.7 |
| 15 | 22 | 61 | 64* | 67* | 71 | 4.2 |
| 16 | 23 | 85 | 90* | 92* | 90 | 6.7 |

*Interpolated from data.

1. A controlled release drug dosage form comprising a core and a coating around said core wherein:
(a) said core comprises a drug-containing composition, another drug-containing composition, and a water-swellable composition, each occupying separate regions within said core, said water-swellable composition being located between said drug-containing composition and said another drug-containing composition; and
(b) said coating is water-permeable, water-insoluble, and has at least one delivery port for communication with said drug-containing composition and another delivery port for communication with said another drug-containing composition.

2. A controlled release drug dosage form comprising a core and a coating around said core wherein:
(a) said core comprises a drug-containing composition and a water-swellable composition, each occupying separate regions within said core, said drug-containing composition surrounding said water-swellable composition;
(b) said drug-containing composition comprises a low-solubility drug and a drug-entraining agent;
(c) said water-swellable composition comprises a swelling agent; and
(d) said coating is water-permeable, water-insoluble, and has at least one delivery port therethrough.

3. A controlled release drug dosage form comprising a core and a coating around said core wherein:
(a) said core comprises a drug-containing composition and a water-swellable composition, each occupying separate regions within said core, said water-swellable composition comprising a plurality of granules;
(b) said drug-containing composition comprises a low-solubility drug and a drug-entraining agent;
(c) said water-swellable-composition comprises a swelling agent; and
(d) said coating is water-permeable, water-insoluble, and has at least one delivery port therethrough.

4. A controlled release drug dosage form comprising a core and a coating around said core wherein:
(a) said core is substantially homogeneous throughout and comprises a mixture of a low-solubility drug, a drug-entraining agent, and a swelling agent; and
(b) said coating is water-permeable, water-insoluble, and has at least one delivery port therethrough.

5. The dosage form of claim 1 wherein said drug-containing composition has a different formulation than said another drug-containing composition.

6. The dosage form of claim 1 wherein said drug-containing composition comprises a low-solubility drug, and said first drug-containing composition comprises a drug-entraining agent.

7. The dosage form of any one of claims 2-4 and 6 wherein said drug-entraining agent is selected from the group consisting of polyols, oligomers of polyethers, mixtures of polyfunctional organic acids, cationic materials, polyethylene oxide, hydroxyethyl cellulose, hydroxypropyl cellulose, hydroxypropylmethyl cellulose, methyl cellulose, carboxyethylcellulose, gelatin, and xanthan gum.

8. The dosage form of any one of claims 1-3 wherein said drug-containing composition further comprises a swelling agent.

9. The dosage form of any one of claims 1-4 wherein said core further comprises a solubilizing agent.

10. The dosage form of any one of claims 1-3 wherein said drug-containing composition further comprises a fluidizing agent having a solubility of at least 30 mg/mL and said fluidizing agent comprises at least 10 wt of said drug-containing composition, and said fluidizing agent is selected from the group consisting of an organic acid, a salt, a sugar, an amino acid, a polyol, and a low-molecular weight oligomer of a water-soluble polymer.

11. The dosage form of any one of claims 14 comprising an ionic swelling agent.

12. The dosage form of any one of claims 1-3 wherein said water-swellable composition has a swelling ratio of at least 2.

13. The dosage form of any one of claims 2-4 and 6 wherein said low-solubility drug is selected from the group consisting of sildenafil and pharmaceutically acceptable salts of sildenafil, sertraline and pharmaceutically acceptable salts of sertraline, the mesylate salt of the drug 4-[3-[4-(2-methylimidazol-1-yl) phenylthio]phenyl]-3,4,5,6-tetrahydro-2H-pyran-4-carboxamide hemifumarate, nifedipine, (+)-2-(3-benzyl-4hydroxy-chroman-7-yl)-4-trifluoromethyl-benzoic acid, 4-amino-5-(4-fluorophenyl)-6,7-dimethoxy-2-[4-(morpholinocarbonyl) perhydro-1,4-diazepin-1-yl]quinoline, and 5-(2-(4-(3-benzisothiazolyl)-piperazinyl)ethyl-6-chlorooxindole.

14. The dosage form of any one of claims 1-4 wherein said coating has a water flux (40/75) of at least 1.0×10⁻³ gm/cm²−hr.

15. The dosage form of any one of claims 14 and 14 wherein said coating has a durability of at least 1 Kp/cm².

16. The dosage form of any one of claims 1-4 wherein said coating is formed from a solution having a weight ratio of cellulose acetate to polyethylene glycol of from 9:1 to 6.5:3.5.

17. The dosage form of any one of claims 14 wherein said coating comprises a polymeric asymmetric membrane comprising a thick, porous region and a dense thin region.

18. The dosage form of any one of claims 2-4 and 6 wherein, following introduction of said dosage form to a use environment, no more than 50 wt % of said low-solubility drug is released to said use environment within 2 hours and at least 60 wt % to said use environment is released within 12 hours.

19. The dosage form of any one of claims 2-4 and 6 wherein, following introduction of said dosage form to a use environment, at least about 80 wt %/o of said low-solubility drug is released to said use environment within about 24 hours.

20. The dosage form of any one of claims 1-4 wherein said core further comprises a concentration-enhancing polymer.
